SEQUENCE LISTING (34 sequences)

SEQ ID NO: 1 - DNA, 40 bp, Homo sapiens
gccatcttacagcctgtttcgagaagacacatcactgcct

SEQ ID NO: 2 - DNA, 40 bp, Homo sapiens
tacagtgtctgaagaagttcgaagtcgctgcagcctaaaa

SEQ ID NO: 3 - DNA, 40 bp, Homo sapiens
ttggccagcaccttgaattcgaacgccttggtctgctcgt

SEQ ID NO: 4 - DNA, 40 bp, Homo sapiens
ttaatttcacaaggcctgcgaattctaatttcatagttgg

SEQ ID NO: 5 - DNA, 40 bp, Homo sapiens
tcatcttctatatctgatcgaacatagcaggaagggttag

SEQ ID NO: 6 - DNA, 40 bp, Homo sapiens
gactgctgtcgatccatacgaattggaaagatcctgattc

SEQ ID NO: 7 - DNA, 40 bp, Homo sapiens
agacagatcaatggttatcgaattgtatataacaatgcag

SEQ ID NO: 8 - DNA, 40 bp, Homo sapiens
caacttagtcaataccatcgaagagatttcctcagggtag

SEQ ID NO: 9 - DNA, 40 bp, Homo sapiens
aactttgcacgacatcaaagaattcataccagagagaagc

SEQ ID NO: 10 - DNA, 40 bp, Homo sapiens
cccctccttaaagaccttcgaatcgtcctgtagcccaaag

SEQ ID NO: 11 - DNA, 40 bp, Homo sapiens
tttcttggcagcatgaattcgatgacttcagcagttccgg

SEQ ID NO: 12 - DNA, 40 bp, Homo sapiens
gaagatgttcaagaaaattcgagggaaagaagtttacatg

SEQ ID NO: 13 - DNA, 40 bp, Homo sapiens
cgctatgtcagaagaaatagaatttatagttccaagtaag

SEQ ID NO: 14 - DNA, 40 bp, Homo sapiens
aaacctggaggaacttttcgaactttctttgtctgtacct

SEQ ID NO: 15 - DNA, 40 bp, Homo sapiens
actaaaatgagaaataattcgattagtagcattacaagct

SEQ ID NO: 16 - DNA, 40 bp, Homo sapiens
tgggagattgacagcaatcgaactgtttgacatactggat

SEQ ID NO: 17 - DNA, 40 bp, Homo sapiens
gcttgaatgtagataagttcgaaaccaaatggctgctagc

SEQ ID NO: 18 - DNA, 40 bp, Homo sapiens
tttctctctcatgtgaattctttcatgtcgtagaaagcaa

SEQ ID NO: 19 - DNA, 40 bp, Homo sapiens
tttttggataggcatgttcgaatccatactggagaaaagc

SEQ ID NO: 20 - DNA, 40 bp, Homo sapiens
ttctgaaacttaacataacgaagtggaacaatgccatctt

SEQ ID NO: 21 - DNA, 40 bp, Homo sapiens
aacctgcatgatcatcagagaattcatactggagaaaaac

SEQ ID NO: 22 - DNA, 40 bp, Homo sapiens
tgtcataaagttgtgaatcgaacttcttgattccggtttt

SEQ ID NO: 23 - DNA, 40 bp, Homo sapiens
gtggagcccataggccatcgaatagatggcgttgatcaca

SEQ ID NO: 24 - DNA, 40 bp, Homo sapiens
gaaactctactgcttgatcgaaatcgtccacagtgacctc

SEQ ID NO: 25 - DNA, 40 bp, Homo sapiens
atctgttgtggagtgccacgaattgtaaataacttcatat

SEQ ID NO: 26 - DNA, 40 bp, Homo sapiens
atccatagtaagtataagagaatttatactggagaggaac

SEQ ID NO: 27 - DNA, 40 bp, Homo sapiens
agaagatcagctggctgtcgaaatggctccgagtcttcac

SEQ ID NO: 28 - DNA, 40 bp, Homo sapiens
agcatgcttaaaaggcttcgaagtaaacttctatcaattg

SEQ ID NO: 29 - DNA, 40 bp, Homo sapiens
tccgcaatcaaagatgttcgaacattttgcatttcttcat

SEQ ID NO: 30 - DNA, 40 bp, Homo sapiens
tgatttaatgagagaaattcgagcgagaaagatggaagac

SEQ ID NO: 31 - DNA, 40 bp, Homo sapiens
agactccaagaagcgaattcgaatggacagttacaccagt

SEQ ID NO: 32 - DNA, 40 bp, Homo sapiens
tattaccggctataaaattcgacacagaaagacgacccgc

SEQ ID NO: 33 - DNA, 40 bp, Homo sapiens
tggaagtctatgtgatcaagaaatcgatagcatttgcagt

SEQ ID NO: 34 - DNA, 40 bp, Homo sapiens
catgacagccatcatcaaagagatcgttagcagaaacaaa

(19) United States
(12) Patent Application Publication
(10) Pub. No.: US 2023/0220446 A1
(43) Pub. Date: Jul. 13, 2023

(54) METHOD AND GENETIC SIGNATURE FOR DETECTING INCREASED TUMOR MUTATIONAL BURDEN

(71) Applicant: Biocartis NV, Mechelen (BE)

(72) Inventors: Jan VAN DE VELDE, Michelen (BE); Bram DE CRAENE, Mechelen (BE); Aleksandra Katarzyna ZWOLINSKA, Mechelen (BE); Hui ZHAO, Gent (Zwijnaarde) (BE); Diether LAMBRECHTS, Gent (Zwijnaarde) (BE); Geert MAERTENS, Mechelen (BE)

(21) Appl. No.: 17/621,552

(22) PCT Filed: Jul. 10, 2020
(86) PCT No.: PCT/EP2020/069639

§ 371 Date: Dec. 21, 2021

(30) Foreign Application Priority Data
Jul. 11, 2019 (EP) 19185822.4

(51) Int. Cl. C12Q 1/6806; C12Q 1/686; C12Q 1/6874

(52) U.S. Cl. CPC C12Q 1/6806; C12Q 1/686; C12Q 1/6874; C12Q 2600/156; C12Q 2600/16

(57) ABSTRACT

The field of the invention generally relates to cancer, including methods for diagnosing, prognosing, and treating cancer. In particular, the field of the invention relates to novel signatures of unique sets of point mutations involving a change of a cytosine or a guanidine, and methods, systems, and components thereof based upon the novel signature for identifying tumor samples having increased tumor mutational burden (TMB). Both the signatures and the methods, systems, and components thereof may be utilized for identifying cancer patients, microsatellite stable-cancer patients in particular, who will effectively respond to immune checkpoint blockade therapy.

### CROSS-REFERENCE TO RELATED APPLICATIONS

This application is the U.S. National Stage of international application PCT/EP2020/069639 filed Jul. 10, 2020, and published as WO 2021/005233 on Jan. 14, 2021, which claims priority to EP Patent Application No. 19185822.4 filed Jul. 11, 2019. The contents of each of the above-referenced applications is incorporated herein by reference in its entirety for all purposes.

### TECHNICAL FIELD

The field of the invention generally relates to cancer, including methods for diagnosing, prognosing, and treating cancer. In particular, the field of the invention relates to novel signatures of unique sets of point mutations involving a change of a cytosine or a guanidine, and methods, systems, and components thereof based upon the novel signature for identifying tumor samples having increased tumor mutational burden (TMB). Both the signatures and the methods, systems, and components thereof may be utilized for identifying cancer patients, microsatellite stable-cancer patients in particular, who will effectively respond to immune checkpoint blockade therapy.

### BACKGROUND

Treatment with immune checkpoint blockade (ICB) therapy antibodies, such as the ones targeting programmed cell death protein 1 (PD-1), its ligand (PD-L1), and/or cytotoxic T-lymphocyte-associated protein 4 (CTLA-4) was shown to potentially result in impressive response rates and durable disease remission, but unfortunately only in a subset of cancer patients. Furthermore, many of the patients that effectively do respond to ICB may experience toxicities (Yuan et al., 2016, J ImmunoTher of Canc). Thus, despite ICB's impressive success in increasing overall survival rates of patients with various types of cancers including metastatic melanoma (Hodi et al., 2010, N Eng J Med), non-small-cell lung cancer (NSCLC) (Borghaei et al., 2015, N Eng J Med), urothelial carcinoma (Rosenberg et al., 2016, Lancet), renal cell carcinoma (Motzer et al., 2015, N Eng J Med), and many others, due to its potentially high toxicity and severe side-effects, there exists a growing need for approaches that may forecast effective responders. At present, this need is even further corroborated by high costs of immunotherapy medications and the reluctance of many medical insurance companies to prepay or refund their prescriptions. For the above reasons, there have been proposed various tests and prediction algorithms to pinpoint responders to ICB.

The detection of PD-L1 by immunohistochemistry (IHC) has been extensively studied as a predictor to anti-PD(L)-1 treatment and is believed to be a valid biomarker in certain settings, as witnessed by a Food and Drug Administration (FDA)-approved companion diagnostic test for pembrolizumab in NSCLC, gastric/gastroesophageal junction adenocarcinoma, cervical cancer and urothelial cancer, and has shown some predictive ability in other cancer types including head and neck cancer and small cell lung carcinoma. However, PD-L1 IHC is an imperfect marker and in many settings it was regarded as inconclusive for prediction of immunotherapy response (Chan et al., 2018, Annals Onc & references therein). For this reason, alternative biomarkers have been evaluated including presence of tumor-infiltrating lymphocytes (TILs) (Tumeh et al., 2014, Nature), T-cell-inflamed gene expression profile (Cristescu et al., 2018, Science), immune gene expression signatures, or even assessment of gut microbiome (Routy et al., 2018, Science; Gopalakrishnan et al., 2018, Science).

It is known now that cancer is a genetic disease wherein accumulation and selection of somatic mutations drive tumor growth and evolution (Hanahan and Weinberg, 2011, Cell). The problem is that every cancer type and even every individual cancer has a unique genetic profile (Ciriello et al., 2013, Nat Gen) and despite frequent prevalence of detectable driver mutations such as those in the KRAS, BRAF, or EGFR genes, which are targetable on their own by specific approaches, their detection usually does not predict how effectively a cancer will respond to the activation of the patient's immune system by ICB.

Accumulating evidence shows that a particularly potent class of antigens that allows the immune system to distinguish normal cells from transformed cancer cells and effectively target the latter ones, is formed by peptides entirely absent from the normal human genome; these antigens are commonly termed ‘neoantigens’. For a large group of human tumors without a viral etiology, such neoantigens solely result from the expression of tumor-specific genetic alternations (Schumacher and Schreiber, 2015, Science). However, it is believed that only a minority of somatic mutations in tumor DNA can be translated and processed to be loaded onto major histocompatibility complex (MHC) molecules for presentation on the cancer cell surface, and it appears that even fewer of them are able to be recognized by the T cells (Coulie et al., 2014, Nat Rev Cancer). Consequently, not all neopeptides are de facto immunogenic (Snyder and Chan, 2015, Curr Opin Genet Dev), and, at least in melanoma, it appears that the bulk of the neoantigen-specific T cell response is directed toward peptides that are essentially unique to a given single specific tumor and that, furthermore, they are unlikely to play a major role in cellular transformation (Gubin et al., 2014, Nature). In conclusion, due to this context uniqueness, it is extremely difficult to establish markers for predicting response to ICB based on neoantigen profiling. It is however plausible, and the gathered data confirms this notion, that the more somatic mutations a tumor has accumulated in general, the more T cell-inducing antigens it will be likely to form and present to the immune system. Consequently, the general estimation of the number of somatic genetic mistakes accumulated within the tumor genome is now broadly being recognized as representing a useful estimation of tumor neoantigen load.

In 2018, the importance of this tumor-specific accumulation of genetic mistakes either manifested as presence of Microsatellite Instability (MSI) or increased Tumor Mutational Burden (TMB, also known as Tumor Mutational Load or TML), was acknowledged by the FDA by marking them as good indicators for immunotherapy in several cancers (Goodman et al., 2017, Mol Canc Therap). Importantly, the FDA approval for anti-PD-1 therapy in patients with any, so called, Microsatellite Instability-High (MSI-H) cancer was the first tissue-agnostic drug approval and the first ever FDA-approved companion biomarker assay for pan-cancer therapy. This has notably marked the important paradigm shift in the cancer field from tissue-specific treatment focus to a more global approach that relies on personalized genetic indications and may be applied to virtually all cancers where the indications are present.

MSI is the genome-wide accumulation of numerous DNA replication errors resulting from impaired DNA mismatch repair (MMR) machinery. These errors can be specifically observed as changes in nucleotide number within single and di-nucleotide repeat sequences, for example (A)ₙ or (CA)ₙ, due to a deletion or an insertion (aka an “indel”) of the repeating unit. It is observed in a substantial subset of colorectal carcinoma (CRC) cases, wherein deficiencies in MMR genes are known to be pivotal for tumorigenesis and disease progression. In fact, the discovery of a single super-responder suffering from an MSI-H CRC quickly led to the successful clinical trials of pembrolizumab in patients with MSI-H or MMR-deficient solid tumors and the rapid approval of pembrolizumab in this biomarker-defined (and not tissue-defined, as it used to be the case before) group of patients (Le et al., 2015, N Eng J Med; Le et al., 2017, Science).

The reliability of MSI-H as an indicator for effective immunotherapy has further been supported by the finding that the MSI-specific increased accumulation of indel-type mutations in the genome correlates with the generation of novel open reading frames encoding neoantigenic sequences (Turajlic et al., 2017, Lancet). The latter may explain why MSI-H tumors naturally exhibit high lymphocytic infiltration, and consequently, select for expression of increased levels of at least five immune checkpoint molecules (Llosa et al., 2014, Canc Discov), which are the exact targets for the therapeutic checkpoint inhibitors. This, and the fact that there exist tests and diagnostic standards available for MSI detection in tumors, including e.g. the initial Bethesda panel and its derivatives, or a more recent and extremely sensitive and fast DNA-based Idylla™ MSI Assay by Biocartis NV that is based on novel short homopolymeric markers (described in PCT/EP2013/057516 and PCT/EP2019/051515), has brought MSI to the present position of a recommended first-line screening tool not only for colorectal and endometrial cancers where MSI-H tumors occur relatively frequently, but also for many other cancer types.

Another histopathological characteristic of many MSI-H tumors is a generally increased Tumor Mutation Burden or Load (TMB or TML). TMB is an extremely interesting phenomenon that stems from the selection by tumors to disable DNA surveillance pathways, which may be different than MMR. Consequently, it is being observed in many cancers that are microsatellite-stable (MSS), notably in melanomas and non-small-cell lung carcinomas (NSCLCs). For example, although the majority of patients with MSI-H solid tumors also have a high TMB, it was estimated that only 16% of patients with high TMB are MSI-H (Chalmers et al., 2017, Genome Med). Importantly, TMB is believed to also represent a very useful estimation of neoantigen load and, hence, to have a huge potential for identifying patients, in particular the ones suffering from MSS tumors with high TMB that cannot be identified by MSI-testing, who will still effectively benefit from immunotherapy (Rizvi et al., 2015, Science; Hugo et al., 2016, Cell).

For example, MSI-H is extremely rare in NSCLC where elevated TMB is relatively frequently observed, although not being as high as the median number of mutations in MSI-H tumors, which often reach thousands per exome (Middha et al., 2017, JCO Precis Oncol). Comparison of findings in small-cell lung cancer (SCLC), NSCLS, and urothelial carcinoma indicates that the TMB threshold for selecting good responders for ICB is about 200 missense mutations, which corresponds to ≥10 mutations per megabase (mut/Mb) by Foundation One testing or to ≥7 mut/Mb by MSK-IMPACT testing (Antonia et al., 2017, World Conf on Lung Canc; Abstract OA 07.03a; Kowanetz rt al., 2016, Ann Oncol; Powles et al., 2018, Genitourinary Canc Symp). Interestingly, applying higher thresholds of TMB equal to 16.2 mut/Mb for atezolizumab treatment (Kowanetz et al., J Thoracic Oncol) or 15 mut/Mb for ipilimumab/nivolumab treatment (Ramalingam et al., 2018, AACR Ann Meeting, Abstract #1137) in NSCLC did not increase the efficacy, which hints to functional background of the selection of ICB-responsive antigens in the tumors. In view of the above, TMB increase in MSS tumors does not have to be massive to identify good responders, although indications exist supperting higher probability of displaying immune-effective neoantigens with higher TMBs (Segal et al., 2008, Cancer Res).

One of the current main challenges in the cancer therapy field for setting exact TMB thresholds to define ICB responders is that, depending on the service provider and their TMB-estimation method used, the TMB counts will substantially differ. Initially, TMB was determined by whole exome sequencing (WES) on tumor DNA matched to normal DNA in order to filter out germline variations and capture exclusively the tumor-acquired somatic mutations (Li et al., 2017, J Mol Diagn). The results are reported as total number of somatic mutations and may, or may not, include indels. WES is still believed to be the best way of measuring exonic TMB but, unfortunately, due to its costs and complexity, it still remains a research only investigation tool that in clinical practice is replaced by more or less exact approximation approaches. For example, a common approach in clinic includes use of targeted NGS panels like F1CDx panel from Foundation Medicine or MSKCC MSK-IMPACT panel, both of which have demonstrated predictive ability for ICB in various published studies and have consequently been approved by the US FDA. F1CDx defines TMB as the total number of synonymous and non-synonymous mutations/megabase (mut/Mb) based on the number of substitutions captured in the coding parts of the panel genes after applying various filters and other mathematical functions, e.g. including filtering out germline events by comparison to public and private variant databases. MSK-IMPACT focuses on non-synonymous mutations using data from sequencing the panel genes from both tumor and germline DNA. There exist more approaches and all of them differ in variables like genomic sizes covered by NGS target gene panels, sequencing depths, mutation types covered, lengths of the reads, cut-points or filters and other mathematical functions applied during variant calling, choice of aligners etc. As a consequence of this variability, the final reported TMB levels will inevitably and frequently very substantially vary depending on the estimation method used.

The above, and the fact that in addition several preanalytical factors (including sample fixation artifacts and NGS library preparation strategy etc.) are likely to affect the final reporting of the TMB counts, there currently exists a large inconsistency in TMB assessment, especially in the potentially clinically-relevant lower TMB ranges. Consequently, setting a uniform and generally-applicable meaningful threshold for TMB classification is currently close to impossible. A desirable alternative could be direct testing for the presence of mutations in genes, which directly cause the TMB-phenotype. Unfortunately, the present state of knowledge about all the possible underlying mechanisms is likely insufficient to define all the genes possibly involved in the process, not to mention that even in the genes which we believe are involved, there is still a lot of information missing about the exact mutations that cause the phenotype. For example, in addition to the mechanisms involved in maintaining DNA replication fidelity, including the p53 pathway or polymerases ε and δ (Korona et al., 2011, Nucl Acids Res; Skoneczna et al. 2015, FEMS Microbiol Rev); DNA proofreading machinery, the afore-mentioned MMR, there exists a plethora of other factors reportedly causative to TMB, from UV light in melanomas, tobacco carcinogens in NSCLC (Jamal-Hanjani et al., 2017, N Engl J Med), to mutations related to APOBEC cytidine deaminase family (McGranahan et al., 2016, Science), or the ones occurring following cytotoxic chemotherapy in resistant emergent tumor subclones (Murugaesu et al., 2015, Cancer Discov). Consequently, given the expected multi-factor nature and complexity of the TMB-related underlying pathways and the exact causative mutations involved (Chalmers et al., 2017, Genome Med), the field would greatly benefit from a provision of a more-tangible and defined “hotspot” signature for capturing even a fraction of TMB-affected immunotherapy-responders, similar to the principle of the existing tests for MSI.

To address the above-discussed shortcomings, we hereby propose for the first time a panel and the methods based thereupon to capture at least a fraction of patients with an increased tumor mutational burden who may still benefit from ICB or other immunotherapy approaches. An advantage of the proposed herein methods is that they capture tumor samples showing a genomic scarring signature reminiscent of a deficiency in POLE gene function (encoding for the catalytic subunit of polymerase E) in microsatellite stable (MMS) patients, who likely may be missed by the existing standard assays like the MSI/MMR-deficiency assays or their complementary tests directed to specific hotspot POLE/POLD1 mutations. In addition, the here presented signature also captures cases with increased TMB that may have originated from perturbations in other repair mechanisms such as mutations in the EXO1 and MUTYH genes. Furthermore, cases with elevated TMB are detected which do not show any apparent underlying mechanism of repair deficiency. These and other features and advantages are explained further herein.

### SUMMARY

Disclosed herein are methods, systems, and components thereof for analyzing the presence of an increased tumor mutational burden (TMB) in a sample obtained from a patient. The disclosed methods and systems typically are utilized for testing at least four different genomic sites as mapped to GRC37 human genome assembly in Table 1 for a presence of a change of a cytosine or a guanine to any other nucleobase, and wherein detection of a presence of at least one of the changes is indicative of a presence of an increased tumor mutational burden (TMB).

The disclosed methods, systems, and components may further be utilized to treat a patient, such as a cancer patient having an increased tumor mutation burden as defined herein. Treatment methods may include administering immunotherapy such anti-PD1, anti-PD-L1, and/or anti cytotoxic T-lymphocyte-associated protein 4 (CTLA-4) therapy, administering chemotherapy, administering radiotherapy, and/or performing surgery or resection of tumor tissue in the patient.

As an example, we present methods, systems, and components for analyzing the presence of an increased tumor mutational burden (TMB) in a sample obtained from a patient. The methods, systems, and components involve testing said sample for a presence of a change of a cytosine or a guanine to any other nucleobase, such as adenine or thymine, in a genomic test site. In some embodiments, the disclosed methods, systems, and components involve testing said sample for the presence of the change in at least four different genomic sites as mapped to GRC37 human genome assembly and listed in Table 1, such as:

chr10 89720744, positioned within PTEN gene;
chr7 112461939, positioned within BMT2 gene;
chr12 89985005, positioned within ATP2B1 gene; and.
chr17 29677227, positioned within NF1 gene,
wherein detection of a presence of at least one change of a cytosine or a guanine is indicative of a presence of an increased tumor mutational burden (TMB).

The sample may be tested for the presence of the change in at least one of the different genomic sites by reacting the sample with reagents that determine the identity of a nucleotide at the different genomic sites. Suitable reagents may include, but are not limited to, primers that hybridize at sequences flanking the site of the change and which can be used to amplify and prepare a polynucleotide sample comprising the change. In some embodiments, the primers may be utilized to prepare amplicons comprising the site of the change and having a size of at least about 50, 100, 150, 200, or 250 nucleotides in length (or having a size within a range bounded by any of these values such as 50-150 nucleotides in length).

Suitable reagents may comprise a primer for sequencing a nucleotide sample and identifying a nucleotide at the different genomic sites. Suitable primers may hybridize at a position flanking the site of the change of a cytosine or a guanine, such as at a position about 10, 20, 30, 40, 50, 60, 70, 80, 90, or 100 nucleotides upstream (or downstream) of the change (or at a position within a range bounded by any of these values such as 10-50 nucleotides upstream or downstream of the change).

In further examples, the disclosed methods, systems, and components involve testing the sample for the presence of changes of a cytosine or a guanine at additional genomic sites as disclosed herein, which may be indicative of increased TMB.

In further examples, the disclosed methods, systems, and components involve testing tumor samples in order to determine the MSI status testing of the tumor sample as Microsatellite-Stable (MSS). In a further aspect, the disclosed methods, samples, and components involve testing tumor samples and determining whether the tumor samples comprise or lack a POLE hotspot mutation selected from P286R and V411L.

The systems disclosed herein may include automated systems that comprise components for performing the methods disclosed herein. Optionally, the disclosed systems comprise an instrument and a cartridge, which are adapted to and/or comprise appropriate structures and/or reagents for performing the methods disclosed herein. Analogously, further are provided cartridges comprising reagents for performing the disclosed methods and operable as part of such automated systems.

In a further aspect, further disclosed are the uses of the disclosed methods, cartridges and systems in TMB detection.

In a yet another but non-limit aspect, additional uses of the herein presented methods, cartridges, and systems are provided in determining if a patient from whom a tumor sample was obtained is to be subjected to a cancer immunotherapy treatment. An example of the latter can be immune checkpoint blockade (ICB) therapy comprising an antibody specific against at least one of the following targets: PD-1, PD-L1, CTLA4, TIM-3, or LAG3. Accordingly, the disclosed methods, systems, and components may involve administering cancer immunotherapy treatment to a patient in need thereof.

### BRIEF DESCRIPTION OF FIGURES

For a fuller understanding, reference is made to the following detailed description taken in conjunction with the accompanying drawings in which:

FIG. 1: shows TMB for TCGA-UCEC tumors in different categories. Red circle indicates the 3 samples having POLD1 mutations but not POLE mutations;

FIG. 2: shows TMB for TCGA-COAD tumors in different categories. The 3 POLD1-mutated samples have base-line TMB;

FIG. 3: shows TMB for TCGA-COAD tumors in different categories. The 3 POLD1-mutated samples have base-line TMB;

FIG. 4: shows TMB for TCGA-non-UCEC and non-COAD tumors in different categories;

FIG. 5: shows TMB for TCGA-UCEC tumors in different categories. The circle indicates covers 8 MSS POLE-non-hotspot-mutated samples identified by retrospective application of the initial 34 marker panel to all UCEC samples in TCGA;

FIG. 6: shows the co-occurrence between the 34 initially identified markers, e.g. RB1CC1 and BRWD3 have a co-occurrence of 1; and lastly

FIG. 7: shows a distribution histogram for 10,000 randomly selected subsets of 4 markers in function of their ability to retrieve samples in the dataset. For a randomly selected 4-marker panel, the maximum number of samples observed is 43 one time, the median being 30.

### DETAILED DESCRIPTION

The practical applications as described herein are based on the identification of a marker panel for detecting signature of POLE-functional-deficiency, which is capable of identifying tumor samples having increased tumor mutational burden (TMB), and therefore also of providing an indication if the patient from whom the tumor sample was derived, may respond effectively to cancer immunotherapy, such as the immune checkpoint blockade (ICB) immunotherapy. An advantage of the herein presented marker panels and methods stems from the fact that they appear to effectively identify samples having an increased TMB even if such samples are microsatellite-stable (MSS) and/or are missing a hotspot POLE mutation. Consequently, the presented herein panels and methods can be seen as opening a gateway for identifying at least a number of patients that can benefit from ICB but are missed by other currently available screening tests.

The herein presented panels are based on initial identification of 34 highly recurrent genetic variants from MSS POLE-hotspot confirmed endometrial cancer (UCEC) records available from whole exome sequencing (WES) results listed in the TCGA database. The 34 recurrent variants involve a change (i.e. mutation) of a cytosine or a guanine to thymine or adenine or possibly any other nucleobase and are listed in the provided herein below Table 1, where they are defined by their positions (“sites”, as further used herein) by reference to the GRCh37/hg19 Human Genome Assembly (currently accessible via e.g. UCSC Genome Browser https://genome.ucsc.edu/). For clarification, when referring to a group or a panel or at least one or more of the hereby disclosed 34 recurrent variants (or, simply, “variants”), different synonymous terms may be used herein in line with their standard meaning as used in the field of molecular biology and biotechnology. These synonymous terms include reference to any one “mutation” or “mutations” (both of the latter possibly with a descriptive e.g. “recurrent mutations”, “newly-identified mutations”, “hereby-disclosed mutations” etc.), “marker” or “markers” (both of the latter possibly with a descriptive), “site of a change of a cytosine or a guanine” or “sites of changes of a cytosine or a guanine” (both of the latter possibly with a descriptive), “change of a cytosine or a guanine” or “changes of a cytosine or a guanine” (both of the latter possibly with a descriptive), or, simply, “change” or “changes” (both of the latter possibly with a descriptive). For the better defining of these newly-identified mutations, Table 1 also provides the name of the gene in which the site of the change that defines the variant is positioned, and the type of the mutation the change causes in the gene product. For example, “stopgain” refers to the type of the mutation that results in a premature termination codon, i.e. wherein “a stop was gained”, which signals the end of translation. Then, the type of the mutation marked as “nonsynonymous SNV” refers to a single nucleotide variant (SNV) that is caused by a missense mutation, i.e. a nucleobase mutation that changes a codon such that a different amino-acid in the product protein is created. Further, Table 1 specifies the exact nucleobase or nucleotide (nt) mutation change in the coding sequence (CDS) of the gene (starting from the START codon of the most common mRNA variant), the amino acid (aa) mutation in the protein product of the gene (“X” indicating truncation), and, in the last column, the wild-type (WT) genomic sequence flanking the site where the mutation occurs (the nt at the site of the change is marked in bold). As used herein, the terms nucleobase and nucleotide can be regarded as largely synonymous and referring to a biochemical unit within a nucleic acid, which can undergo a mutational change. The tiny nuance in their meaning is that from purely biochemical perspective, a nucleobase is a nitrogenous heterocyclic base of a nucleic acid, which can either be a double-ringed purine, such as adenine (A) or guanine (G), or a single-ringed pyrimidine, such as thymine (T), uracil (U), cytosine (C). Conversely, a nucleotide is the actual monomer that builds a nucleic acid biopolymer molecule strand, e.g. of DNA or RNA, wherein each nucleotide consists of the nucleobase, a five-carbon pentose sugar (deoxyribose in DNA or ribose in RNA), and a phosphate group. In the last column of Table 1, the WT base at the mutated variant position is always presented at the nucleotide no. 20, i.e. there are 19 nt (nucleotides) provided upstream and 20 nt provided downstream of the change site. Remarkably, as can be seen from the column detailing the nt change in the CDS, the affected nucleobase is always cytosine (C) or its complementary pairing nucleobase guanine (G). Even more remarkably, all of the recurrent variants consist of a C or a G mutation in a very similar sequence context. Namely, 33 out of 34 identified recurrent variants occur within a trinucleotide sequence TTC or its complementary GAA (sequences always provided in 5′->3′ direction, nucleotides that become mutated in the recurrent variants are underlined). Furthermore, 23 of them occur within the same 5-nt strip sequence of TTCGA or its complement TCGAA (the change sites underlined). The finding is consistent with previous reports about POLE deficiency mutational patterns (Shinbrot et al., 2014, Genome Res) and highlights the specificity of the identified herein variants for a POLE scarring signature. Interestingly, 79.4% of the changes concern change of cytosine to thymine (C->T, which in DNA is the same as change of guanine to adenine, G->A, depending on which DNA strand given mutation is read), while the remaining 20.6% concern C->A or G->T (depending on which DNA strand the mutation is read).

TABLE 1

| | | | | | | mutation position flanking |
|---|---|---|---|---|---|---|
| SEQ | | | | nt change | mutation | region (WT sequence; |
| ID | position in | gene | mutation | in CDS | in gene | mutation position at |
| NO. | GRCh37/hg19 | name | type | vis. WT | product | nt no. = 20 marked in bold) |
| 1 | chr19 47424921 | ARHGAP35 | stopgain | C2989T | R997X | >chr19:47424901-47424941 |
| | | | | | | GCCATCTTAC AGCCTGTTTC |
| | | | | | | GAGAAGACAC ATCACTGCCT |
| 2 | chr17 29677227 | NF1 | stopgain | C7348T | R2450X | >chr17:29677207-29677247 |
| | | | | | | TACAGTGTCT GAAGAAGTTC |
| | | | | | | GAAGTCGCTG CAGCCTAAAA |
| 3 | chrX 99662008 | PCDH19 | non- | G1588A | E530K | >chrX:99661988-99662028 |
| | | | synonymous | | | TTGGCCAGCA CCTTGAATTC |
| | | | SNV | | | GAACGCCTTG GTCTGCTCGT |
| 4 | chr9 5968511 | KIAA2026 | non- | C1720T | R574C | chr9:5968491-5968531 |
| | | | synonymous | | | TTAATTTCAC AAGGCCTGCG |
| | | | SNV | | | AATTCTAATT TCATAGTTGG |
| 5 | chr7 112461939 | BMT2 | stopgain | C1078T | R360X | >chr7:112461919-112461959 |
| | | | | | | tcatcttcta tatctGATCG |
| | | | | | | AACATAGCAG GAAGGGTTAG |
| 6 | chrX 74519615 | UPRT | non- | G608A | R203Q | >chrX:74519595-74519635 |
| | | | synonymous | | | GACTGCTGTC GATCCATACG |
| | | | SNV | | | AATTGGAAAG ATCCTGATTC |
| 7 | chr8 121228689 | COL14A1 | non- | G1697A | R566Q | >chr8:121228669-121228709 |
| | | | synonymous | | | AGACAGATCA ATGGTTATCG |
| | | | SNV | | | AATTGTATAT AACAATGCAG |
| 8 | chr6 31779382 | HSPA1L | non- | C368T | S123L | >chr6:31779362-31779402 |
| | | | synonymous | | | CAACTTAGTC AATACCATCG |
| | | | SNV | | | AAGAGATTTC CTCAGGGTAG |
| 9 | chr19 52825339 | ZNF480 | non- | G707T | R236I | >chr19:52825319-52825359 |
| | | | synonymous | | | AACTTTGCAC GACATCAAAG |
| | | | SNV | | | AATTCATACC AGAGAGAAGC |
| 10 | chr13 47409732 | HTR2A | non- | C404T | S135L | >chr13:47409712-47409752 |
| | | | synonymous | | | CCCCTCCTTA AAGACCTTCG |
| | | | SNV | | | AATCGTCCTG TAGCCCAAAG |
| 11 | chr11 60468341 | MS4A8 | non- | C8T | S3L | >chr11:60468321-60468361 |
| | | | synonymous | | | TTTCTTGGCA GCATGAATTC |
| | | | SNV | | | GATGACTTCA GCAGTTCCGG |
| 12 | chrX 110970087 | ALG13 | stopgain | C1468T | R490X | >chrX:110970067-110970107 |
| | | | | | | GAAGATGTTC AAGAAAATTC |
| | | | | | | GAGGGAAAGA AGTTTACATG |
| 13 | chr3 370022 | CHL1 | stopgain | G370T | E124X | >chr3:370002-370042 |
| | | | | | | CGCTATGTCA GAAGAAATAG |
| | | | | | | AATTTATAGT TCCAAGTAAG |
| 14 | chr18 53017619 | TCF4 | stopgain | C520T | R174X | >chr18:53017599-53017639 |
| | | | | | | AAACCTGGAG GAACTTTTCG |
| | | | | | | AACTTTCTTT GTCTGTACCT |
| 15 | chr6 101296418 | ASCC3 | non- | G407A | R136Q | >chr6:101296398-101296438 |
| | | | synonymous | | | ACTAAAATGA GAAATAATTC |
| | | | SNV | | | GATTAGTAGC ATTACAAGCT |
| 16 | chr4 115544340 | UGT8 | non- | G304A | E102K | >chr4:115544320-115544360 |
| | | | synonymous | | | TGGGAGATTG ACAGCAATCG |
| | | | SNV | | | AACTGTTTGA CATACTGGAT |
| 17 | chr2 9098719 | MBOAT2 | non- | G128A | R43Q | >chr2:9098699-9098739 |
| | | | synonymous | | | GCTTGAATGT AGATAAGTTC |
| | | | SNV | | | GAAACCAAAT GGCTGCTAGC |
| 18 | chr19 12501557 | ZNF799 | non- | G1655T | R552I | >chr19:12501537-12501577 |
| | | | synonymous | | | TTTCTCTCTC ATGTGAATTC |
| | | | SNV | | | TTTCATGTCG TAGAAAGCAA |
| 19 | chr18 74635035 | ZNF236 | non- | G3560A | R1187Q | >chr18:74635015-74635055 |
| | | | synonymous | | | TTTTTGGATA GGCATGTTCG |
| | | | SNV | | | AATCCATACT GGAGAAAAGC |
| 20 | chr18 54281690 | TXNL1 | non- | C700T | R234C | >chr18:54281670-54281710 |
| | | | synonymous | | | TTCTGAAACT TAACATAACG |
| | | | SNV | | | AAGTGGAACA ATGCCATCTT |
| 21 | chr16 68598492 | ZFP90 | non- | G1802T | R601I | >chr16:68598472-68598512 |
| | | | synonymous | | | AACCTGCATG ATCATCAGAG |
| | | | SNV | | | AATTCATACT GGAGAAAAAC |
| 22 | chr12 89985005 | ATP2B1 | non- | C3419T | S1140L | >chr12:89984985-89985025 |
| | | | synonymous | | | TGTCATAAAG TTGTGAATCG |
| | | | SNV | | | AACTTCTTGA TTCCGGTTTT |
| 23 | chr11 88338063 | GRM5 | non- | C1217T | S406L | >chr11:88338043-88338083 |
| | | | synonymous | | | GTGGAGCCCA TAGGCCATCG |
| | | | SNV | | | AATAGATGGC GTTGATCACA |
| 24 | chr10 128908585 | DOCK1 | non- | G2590A | E864K | >chr10:128908565-128908605 |
| | | | synonymous | | | GAAACTCTAC TGCTTGATCG |
| | | | SNV | | | AAATCGTCCA CAGTGACCTC |
| 25 | chr1 78428511 | FUBP1 | non- | C1351T | R451C | >chr1:78428491-78428531 |
| | | | synonymous | | | ATCTGTTGTG GAGTGCCACG |
| | | | SNV | | | AATTGTAAAT AACTTCATAT |
| 26 | chr1 227843477 | ZNF678 | non- | G1691T | R564I | >chr1:227843457-227843497 |
| | | | synonymous | | | ATCCATAGTA AGTATAAGAG |
| | | | SNV | | | AATTTATACT GGAGAGGAAC |
| 27 | chrX 79942391 | BRWD3 | stopgain | C3976T | R1326X | >chrX:79942371-79942411 |
| | | | | | | AGAAGATCAG CTGGCTGTCG |
| | | | | | | AAATGGCTCC GAGTCTTCAC |
| 28 | chrX 119678368 | CUL4B | stopgain | C1105T | R369X | >chrX:119678348-119678388 |
| | | | | | | AGCATGCTTA AAAGGCTTCG |
| | | | | | | AAGTAAACTT CTATCAATTG |
| 29 | chr8 53558288 | RB1CC1 | stopgain | C3961T | R1321X | >chr8:53558268-53558308 |
| | | | | | | TCCGCAATCA AAGATGTTCG |
| | | | | | | AACATTTTGC ATTTCTTCAT |
| 30 | chr7 39745749 | RALA | stopgain | C526T | R176X | >chr7:39745729-39745769 |
| | | | | | | TGATTTAATG AGAGAAATTC |
| | | | | | | GAGCGAGAAA GATGGAAGAC |
| 31 | chr2 113417110 | SLC20A1 | stopgain | C1378T | R460X | >chr2:113417090-113417130 |
| | | | | | | AGACTCCAAG AAGCGAATTC |
| | | | | | | GAATGGACAG TTACACCAGT |
| 32 | chr18 50832017 | DCC | stopgain | C1981T | R661X | >chr18:50831997-50832037 |
| | | | | | | TATTACCGGC TATAAAATTC |
| | | | | | | GACACAGAAA GACGACCCGC |
| 33 | chr10 89720744 | PTEN | stopgain | G895T | E299X | >chr10:89720724-89720764 |
| | | (″PTEN(i)″) | | | | TGGAAGTCTA TGTGATCAAG |
| | | | | | | AAATCGATAG CATTTGCAGT |
| 34 | chr10 89624245 | PTEN | stopgain | G538T | E180X | >chr10:89624225-89624265 |
| | | (″PTEN | | | | CATGACAGCC ATCATCAAAG |
| | | (ii)″) | | | | AGATCGTTAG CAGAAACAAA |

The recurrent 34 changes of a cytosine or a guanine as initially identified in TCGA-MSS-UCEC samples were then tested against all tumor records in the TCGA database, the details of which are explained in continuation in the Examples section. As a result of this analysis, 82 samples from different tumors were retrieved, which details are provided in Table 2 (wherein “MSS”=microsatellite stable; “MSI-L” or “MSI-H”=MSI positive; “Hotspot”—POLE hotspot mutation present; “POLE”=POLE non-hotspot mutation present; “EXO1”=EXO1 mutation present; “MUTYH”=MUTYH mutation present, “NA”=data not available, i.e. presence of the mutation of interest not indicated in TCGA; TMB expressed as substitutions/Mb, not containing indels).

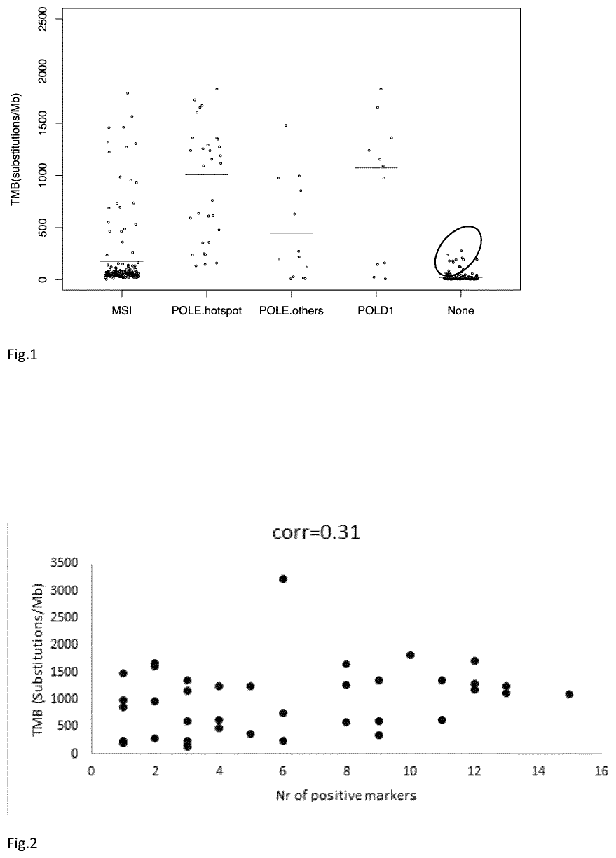

Interestingly, 56 of these samples were annotated in TCGA as having TMB>300 substitutions/megabase (subst/Mb), which we labelled as having a hyper-mutator phenotype or hyperTMB (“HYPER)”. Further, 64 had TMB>200 subst/Mb (upper-end high TMB or “high+” and above), 72 had TMB>100 subst/Mb (medium-range high or “high” and above), and 7 had TMB<50 subst/Mb (classified by us as having a medium and low increment in TMB; “med incr” and “low incr”). 55 of the samples were MSS, 66 had a mutation in POLE gene (out of which 44 samples had a POLE hotspot mutation), 6 were positive for EXO1 mutation, while 4 were positive for MUTYH mutation. All of the above suggests a promising specificity for detecting samples with perturbations in any of DNA surveillance mechanisms, and in particular, the ones that cannot be detected by MSI tests or tests directed to hotspot POLE mutations. Of note, the markers of the panel appear surprisingly efficient in identifying high and in particular hyperTMB-affected samples, that in most cases are MSS samples, which has a huge potential for the identification of the fraction of effective-responders to ICB, who would otherwise be missed by the current screening tests. Especially that there appears to be no correlation between the number of mutated variants and the level of TMB (shown later in FIG. 2), which means that each mutated marker on its own can already be a predictor of an increased TMB present in the sample.

TABLE 2

| # | PatientID | Cancer | nrPos | TMB | Class | MSI | POLE | EXO1 | MUTYH |
|---|---|---|---|---|---|---|---|---|---|
| 1 | TCGA-A5-A0G2 | UCEC | 6 | 3217.9 | HYPER | MSI-L | Hotspot | NA | NA |
| 2 | TCGA-FW-A3R5 | SKCM | 3 | 1891.5 | HYPER | MSS | NA | EXO1 | NA |
| 3 | TCGA-AG-A002 | READ | 7 | 1846.8 | HYPER | MSS | POLE | NA | MUTYH |
| 4 | TCGA-AP-A0LM | UCEC | 10 | 1826.3 | HYPER | MSS | Hotspot | NA | NA |
| 5 | TCGA-AX-A2HC | UCEC | 1 | 1788.8 | HYPER | MSI-H | POLE | NA | NA |
| 6 | TCGA-EO-A3B0 | UCEC | 12 | 1723.6 | HYPER | MSS | Hotspot | NA | NA |
| 7 | TCGA-EO-A22R | UCEC | 2 | 1669.4 | HYPER | MSI-L | Hotspot | NA | NA |
| 8 | TCGA-E6-A1LX | UCEC | 8 | 1651.6 | HYPER | MSI-L | Hotspot | NA | NA |
| 9 | TCGA-FI-A2D5 | UCEC | 2 | 1603.8 | HYPER | MSS | Hotspot | NA | NA |
| 10 | TCGA-EO-A22U | UCEC | 7 | 1564.1 | HYPER | MSI-H | Hotspot | NA | NA |
| 11 | TCGA-AP-A1DV | UCEC | 1 | 1478.9 | HYPER | MSI-L | POLE | NA | NA |
| 12 | TCGA-B5-A3FA | UCEC | 3 | 1360.5 | HYPER | MSI-L | Hotspot | NA | NA |
| 13 | TCGA-EO-A22X | UCEC | 11 | 1359.7 | HYPER | MSS | Hotspot | NA | NA |
| 14 | TCGA-BS-A0UF | UCEC | 9 | 1346.7 | HYPER | MSS | Hotspot | NA | NA |
| 15 | TCGA-IB-7651 | PAAD | 1 | 1318.3 | HYPER | MSS | Hotspot | EXO1 | NA |
| 16 | TCGA-AX-A1CE | UCEC | 1 | 1310.2 | HYPER | MSI-H | POLE | NA | NA |
| 17 | TCGA-A5-A0G1 | UCEC | 1 | 1304.2 | HYPER | MSI-H | POLE | NA | NA |
| 18 | TCGA-B5-A0JY | UCEC | 12 | 1289.8 | HYPER | MSS | Hotspot | NA | NA |
| 19 | TCGA-A5-A2K5 | UCEC | 8 | 1273.5 | HYPER | MSS | Hotspot | NA | NA |
| 20 | TCGA-AP-A056 | UCEC | 13 | 1255.3 | HYPER | MSI-L | Hotspot | NA | NA |
| 21 | TCGA-B5-A11E | UCEC | 4 | 1238.9 | HYPER | MSI-L | Hotspot | NA | NA |
| 22 | TCGA-BS-A0UV | UCEC | 5 | 1236.7 | HYPER | MSI-L | Hotspot | NA | NA |
| 23 | TCGA-AX-A05Z | UCEC | 12 | 1188.8 | HYPER | MSS | Hotspot | NA | NA |
| 24 | TCGA-06-5416 | GBM | 2 | 1171.3 | HYPER | MSS | Hotspot | EXO1 | NA |
| 25 | TCGA-DF-A2KU | UCEC | 3 | 1154.1 | HYPER | MSI-L | Hotspot | NA | NA |
| 26 | TCGA-AJ-A3EL | UCEC | 13 | 1116.2 | HYPER | MSI-L | Hotspot | NA | NA |
| 27 | TCGA-AX-A0J0 | UCEC | 15 | 1091.8 | HYPER | MSI-L | Hotspot | NA | NA |
| 28 | TCGA-F5-6814 | READ | 12 | 1002.7 | HYPER | MSS | Hotspot | EXO1 | NA |
| 29 | TCGA-AX-A06F | UCEC | 1 | 994.8 | HYPER | MSI-L | POLE | NA | NA |
| 30 | TCGA-AP-A051 | UCEC | 1 | 985.2 | HYPER | MSI-H | POLE | NA | NA |
| 31 | TCGA-D1-A103 | UCEC | 2 | 974.9 | HYPER | MSI-L | POLE | NA | NA |
| 32 | TCGA-CA-6717 | COAD | 3 | 930.4 | HYPER | MSS | POLE | EXO1 | NA |
| 33 | TCGA-AZ-4315 | COAD | 3 | 876.5 | HYPER | MSS | Hotspot | NA | MUTYH |
| 34 | TCGA-B5-A1MR | UCEC | 1 | 852.7 | HYPER | MSS | POLE | NA | NA |
| 35 | TCGA-AA-A00N | COAD | 2 | 797.5 | HYPER | MSI-L | Hotspot | NA | NA |
| 36 | TCGA-D1-A17Q | UCEC | 6 | 760.3 | HYPER | MSI-L | Hotspot | NA | NA |
| 37 | TCGA-AJ-A3EK | UCEC | 2 | 735.6 | HYPER | MSI-H | POLE | NA | NA |
| 38 | TCGA-EO-A3AV | UCEC | 11 | 634.5 | HYPER | MSS | Hotspot | NA | NA |
| 39 | TCGA-AP-A1E0 | UCEC | 4 | 629.4 | HYPER | MSI-L | POLE | NA | NA |
| 40 | TCGA-AN-A046 | BRCA | 7 | 623.8 | HYPER | MSS* | Hotspot | NA | NA |
| 41 | TCGA-EY-A1GI | UCEC | 9 | 613.5 | HYPER | MSS | Hotspot | NA | NA |
| 42 | TCGA-BK-A6W3 | UCEC | 3 | 609.7 | HYPER | MSS | Hotspot | NA | NA |
| 43 | TCGA-19-5956 | GBM | 3 | 596.1 | HYPER | MSS | POLE | NA | NA |
| 44 | TCGA-EO-A3AY | UCEC | 8 | 590.8 | HYPER | MSS | Hotspot | NA | NA |
| 45 | TCGA-BR-8680 | STAD | 1 | 582.8 | HYPER | MSS | Hotspot | NA | NA |
| 46 | TCGA-AA-3984 | COAD | 4 | 581.3 | HYPER | MSS | Hotspot | NA | NA |
| 47 | TCGA-EI-6917 | READ | 6 | 484.5 | HYPER | MSS | Hotspot | NA | MUTYH |
| 48 | TCGA-AJ-A5DW | UCEC | 4 | 476.8 | HYPER | MSS | Hotspot | NA | NA |
| 49 | TCGA-VQ-A8P2 | STAD | 2 | 447 | HYPER | MSI-H | POLE | NA | NA |
| 50 | TCGA-AA-3977 | COAD | 3 | 361.9 | HYPER | MSS | POLE | NA | NA |
| 51 | TCGA-EY-A1G8 | UCEC | 5 | 358.6 | HYPER | MSS | Hotspot | NA | NA |
| 52 | TCGA-DK-A6AW | BLCA | 3 | 355.9 | HYPER | MSS | Hotspot | NA | MUTYH |
| 53 | TCGA-D1-A16X | UCEC | 9 | 354 | HYPER | MSS | Hotspot | NA | NA |
| 54 | TCGA-AA-3510 | COAD | 3 | 352.1 | HYPER | MSS | POLE | NA | NA |
| 55 | TCGA-CA-6718 | COAD | 1 | 332 | HYPER | MSS | Hotspot | NA | NA |
| 56 | TCGA-AG-3892 | READ | 4 | 317.5 | HYPER | MSS | POLE | NA | NA |
| 57 | TCGA-FR-A8YC | SKCM | 1 | 276 | high+ | MSS | NA | EXO1 | NA |
| 58 | TCGA-E6-A1M0 | UCEC | 2 | 272.9 | high+ | MSS | POLE | NA | NA |
| 59 | TCGA-FU-A3HZ | CESC | 2 | 262.1 | high+ | MSS | POLE | NA | NA |
| 60 | TCGA-AJ-A3BH | UCEC | 1 | 260.2 | high+ | MSI-H | POLE | NA | NA |
| 61 | TCGA-A5-A0GP | UCEC | 1 | 247.6 | high+ | MSS | Hotspot | NA | NA |
| 62 | TCGA-B5-A11N | UCEC | 3 | 240.7 | high+ | MSI-L | Hotspot | NA | NA |
| 63 | TCGA-QF-A5YS | UCEC | 6 | 236.3 | high+ | MSS | Hotspot | NA | NA |
| 64 | TCGA-BS-A0TC | UCEC | 1 | 217.5 | high+ | MSS | POLE | NA | NA |
| 65 | TCGA-DF-A2KV | UCEC | 1 | 188.4 | high | MSS | POLE | NA | NA |
| 66 | TCGA-XN-A8T3 | PAAD | 6 | 173 | high | MSS | NA | NA | NA |
| 67 | TCGA-EY-A1GD | UCEC | 3 | 159.7 | high | MSS | Hotspot | NA | NA |
| 68 | TCGA-D1-A16Y | UCEC | 3 | 145.8 | high | MSI-L | Hotspot | NA | NA |
| 69 | TCGA-D3-A5GO | SKCM | 1 | 144.9 | high | MSS | NA | NA | NA |
| 70 | TCGA-QS-A5YQ | UCEC | 3 | 132.4 | high | MSS | Hotspot | NA | NA |
| 71 | TCGA-WE-A8K5 | SKCM | 1 | 107 | high | MSS | NA | NA | NA |
| 72 | TCGA-D3-A51G | SKCM | 1 | 106.5 | high | MSS | NA | NA | NA |
| 73 | TCGA-FR-A3YO | SKCM | 1 | 79.1 | high− | MSS | NA | NA | NA |
| 74 | TCGA-FS-A4F2 | SKCM | 1 | 75.2 | high− | MSS | NA | NA | NA |
| 75 | TCGA-YB-A89D | PAAD | 2 | 56.4 | high− | MSS | NA | NA | NA |
| 76 | TCGA-VQ-A8PB | STAD | 1 | 44.4 | med incr | MSI-H | NA | NA | NA |
| 77 | TCGA-VQ-A91E | STAD | 1 | 43.9 | med incr | MSI-H | NA | NA | NA |
| 78 | TCGA-DM-A28C | COAD | 1 | 15.4 | med incr | MSS | NA | NA | NA |
| 79 | TCGA-33-AASJ | LUSC | 1 | 13.3 | med incr | MSS | NA | NA | NA |
| 80 | TCGA-IN-A6RP | STAD | 1 | 11.6 | med incr | MSS | NA | NA | NA |
| 81 | TCGA-41-3392 | GBM | 1 | 5.3 | low incr | MSS | NA | NA | NA |
| 82 | TCGA-VQ-A8PD | STAD | 1 | 4.9 | low incr | MSS | NA | NA | NA |

The finding of the 34 single nucleotide variants specifically associated with an increased TMB is unexpected. Increased TMB is expected to be caused by deficiencies in DNA replication and repair, and the mutations would be expected to be randomly spread and scattered over the cancer cell genome. Today, the increased TMB needs to be assessed by sequencing of hundreds of amplicons with the coverage of about 1 Mb (Büttner et al., 2019, ESMO Open Canc Horiz), which requires a large sequencing capacity. The finding that each of the 34 SNVs on their own is predictive of an elevated TMB is therefore surprising and point to the 34 loci as preferred targets for the replication and repair deficiencies such as deficient POLE, EXO1, MUTYH, and hitherto unidentified other mechanisms. Since the signature is observed in MSS samples, it is independent of MSI or deficient MMR. Notably, the median TMB level found with the 34 SNVs equals to 612 mut/Mb, which is substantially much higher as compared to median TMB in MSI samples, which was reported to be around 47 mutations/Mb on average (Fabrizio et al., 2018, J Gastrointest Oncol). Furthermore, the number of samples having TMB<10 in TCGA is 3529, out of which two were positive for one of the 34 SNVs of Table 1. This suggests a very high specificity and a strong association of each of the 34 markers with an increased TMB, and consequently, it further advocates for their application in clinical use. Because of the low number of targets, the herein identified markers could be efficiently used for the detection of increased TMB in a variety of diagnostic applications. These notably include a PCR-based detection or the addition of the 34 loci to existing NGS pipelines without the need for much higher NGS capacity in order to identify cancer patients positive for the increased TMB, who are expected to be prime candidates for response to immunotherapy.

In view of the above, methods, systems, and components are provided for analyzing the presence of an increased tumor mutational burden (TMB) in a sample obtained from a patient, the methods, systems, and components, involving classifying the sample as having an increased tumor mutational burden (TMB), if at least one of the genomic sites of Table 1 as mapped to GRC37 human genome assembly contains a change of a cytosine or a guanine to any other nucleobase (for example, a thymine or an adenine), and wherein detection of the presence of at least one of such changes is indicative of an increased tumor mutational burden (TMB). In possible embodiments, the change of a cytosine or a guanine to any other nucleobase is selected from a change of a cytosine to thymine or adenine, and a change of guanine to adenine or thymine. In further embodiments, the change of a cytosine or a guanine to any other nucleobase is selected from a change of a cytosine to thymine and a change of guanine to adenine.

For example, the disclosed methods, systems, and components may involve analyzing for the presence of an increased tumor mutational burden (TMB) in a sample obtained from a patient. In some embodiments, the methods, systems, and components may involve testing at least four different genomic sites as mapped to GRC37 human genome assembly in Table 1 for a presence of a a change of a cytosine or a guanine to any other nucleobase (for example, a thymine or an adenine), and wherein detection of the presence of at least one of the mutations is indicative of an increased tumor mutational burden (TMB). In possible embodiments, the change of a cytosine or a guanine to any other nucleobase is selected from a change of a cytosine to thymine or adenine, and a change of guanine to adenine or thymine. In further embodiments, the change of a cytosine or a guanine to any other nucleobase is selected from a change of a cytosine to thymine and a change of guanine to adenine.

As used herein, the term increased TMB is to be construed as increased tumor mutational burden or tumor mutational load (TMB or TML, respectively) with reference to a normal, i.e. non tumor sample, usually being a normal tissue matched sample from the same patients. As TMB values are greatly depending on the method of their estimation used (WES or target enriched NGS, also depending which mutations and functions are included in the estimations), the exemplary values as provided herein are consistent with the annotations as retrieved from TCGA and include synonymous and non-synonymous substitutions/Mb but do not include indels. With regard to the TMB as defined in the TCGA, it can be assumed that the presented herein methods can indicate presence of an increased TMB defined as showing more than 4.5 substitutions/Mb. However, depending on the variants selected from Table 1 and context-dependent application of various screening thresholds, in possible embodiments, the increased TMB can be defined as showing more than 10 substitutions/Mb/, possibly more than 50 substitutions/Mb, or possibly more than 100 substitutions/Mb. In an embodiment, it can be defined as showing more than 200 or even more than 300 substitutions/Mb.

Exemplary selections of 4 markers from Table 1 allow to cover the following numbers of all samples from Table 2. For PTEN(i), BMT2, ATP2B1 and GRM5 we cover 44/82^{˜}54%, 7 being high, 36 hyper, and 1 being the glioblastoma sample having a low increment TMB. 65% of UCEC samples are covered. For PTEN(i), BMT2, ATP2B1 and NF1, 43 samples are covered from 82 (9 high, 34 hyper). Also 65% UCEC samples are covered. In line with the above and based on estimations of the individual strengths of each and every variant marker, it was found that exemplary four markers that very well perform together are the ones positioned in the BMT2 gene, ATP2B1 gene, NF1 gene, and in the PTEN gene at the position chr10 89720744, further referred to as PTEN(i), due to the identification of two recurrent variants in PTEN.

Hence, in some embodiments, the disclosed methods, systems, and components may involve detecting the change at four or more different genomic sites of Table 1, optionally wherein the at least four different genomic sites from Table 1 are selected from:

chr10 89720744, positioned within PTEN gene;
chr7 112461939, positioned within BMT2 gene;
chr12 89985005, positioned within ATP2B1 gene and.
chr17 29677227, positioned within NF1 gene.

An exemplary selection of a 5-marker panel made of PTEN(i), BMT2, ATP2B1, NF1, and either of GRM5 or UGT8, allows us to retrieve 50/82 samples from Table 2 (^{˜}61%). In detail, panel of PTEN(i), BMT2, ATP2B1, NF1, and GRM5 provide 50/82 coverage, including 9 high, 40 hyper and 1 with low increment (glioblastoma). The UCEC coverage for this combination is 72%. For PTEN(i), BMT2, ATP2B1, NF1, and UGT8, the total coverage is 50/82, 11 high, 39 hyper, and 70% of UCEC. Hence, in another possible embodiment, the disclosed methods, systems, and components involve further testing for the presence of the change at the following site from Table 1: chr11 88338063, positioned within GRM5 gene.

Next, performance of a 6 marker panels including e.g. PTEN(i), BMT2, ATP2B1, NF1+any of GRM5, UTG8, HTR2A, or ZNF678 is the following. For PTEN(i), BMT2, ATP2B1, NF1, GRM5 and UGT8 equals 55/82, 11 high, 43 hyper, and 1 low increased, also 74% UCEC. For PTEN(i), BMT2, ATP2B1, NF1, GRM5 and HTR2A, 55/82, 10 high, 42 hyper, 2 low, 1 med, 78% UCEC. For PTEN(i), BMT2, ATP2B1, NF1, UGT8 and HTR2A, 56/82, 12 high, 42 hyper, 1 low, 1 med, and 78% UCEC. Hence, in a next possible embodiment, the disclosed methods, systems, and components further involve testing for the presence of the change at the following site from Table 1: chr4 115544340, positioned within UGT8 gene.

In further embodiments, the disclosed methods, systems, and components involve further testing for the presence of the change in at least two of the following sites from Table 1:

chr13 47409732, positioned within HTR2A gene;
chr1 227843477, positioned within ZNF678 gene.
The above and other exemplary 7-marker panels have the following coverage (the variant further referred to as PTEN(ii) designates mutation at the site: chr10 89624245, positioned within PTEN gene). NF1 BMT2 ATP2B1 PTEN(i) GRM5 UGT8 HTR2A, 60/82, 12 high. 45 hyper, 2 low, 1 med, and 80% of all UCEC. NF1 BMT2 ATP2B1 PTEN(i) GRM5 UGT8 PTEN(ii), 60/82, 11 high, 47 hyper, 1 low, 1 med, 78% UCEC. NF1 BMT2 ATP2B1 PTEN(i) GRM5 UGT8 ZNF678, 59/82, 12 high, 46 hyper, 1 low, 80% UCEC. NF1 BMT2 ATP2B1 PTEN(i) GRM5 HTR2A PTEN(ii), 59/82, 10 high, 45 hyper, 2 low, 2 med, 80% UCEC. NF1 BMT2 ATP2B1 PTEN(i) GRM5 HTR2A ZNF678, 59/82, 11 high, 45 hyper, 2 low, 1 med, 83% UCEC. NF1 BMT2 ATP2B1 PTEN(i) GRM5 PTEN(ii) ZNF678, 60/82, 10 high, 48 hyper, 1 low, 1 med, 83% UCEC. NF1 BMT2 ATP2B1 PTEN(i) UGT8 HTR2A PTEN(ii), 60/82, 12 high, 45 hyper, 1 low, 2 med, 80% UCEC. NF1 BMT2 ATP2B1 PTEN(i) UGT8 HTR2A ZNF678, 59/82, 13 high, 44 hyper, 1 low, 1 med, 83% UCEC, NF1 BMT2 ATP2B1 PTEN(i) UGT8 PTEN(ii) ZNF678, 60/82, 12 high, 47 hyper, 1 med, 83% UCEC. NF1 BMT2 ATP2B1 PTEN(i) HTR2A PTEN(ii) ZNF678, 58/82, 11 high, 43 hyper, 1 low, 2 med and 80% of all UCEC.

In another embodiment, the disclosed methods, systems, and components further involve testing for the presence of the change at the following site from Table 1: chr10 89624245, positioned within PTEN gene (the variant above and further referred to as PTEN(ii)).

As can be seen from above computations, addition of one marker each time improves coverage of samples from Table 2. We observed that to cover all samples in Table 2 19 markers are sufficient instead of the initial 34 identified. In accordance with this observation, the alternative panels, each time one marker larger than the directly above-described exemplary panels, can be provided as further exemplary embodiments of the invention until a 19-marker panel or larger is achieved covering all the samples from Table 2.

In a next embodiment, the disclosed methods, systems, and components further involve testing for the presence of the change at the following site from Table 1: chr19 47424921, positioned within ARHGAP35 gene.

In another embodiment, the disclosed methods, systems, and components further involve testing for the presence of the change at the following site from Table 1: chr8 121228689, positioned within COL14A1 gene.

In another embodiment, the disclosed methods, systems, and components further involve testing for the presence of the change in the following sites from Table 1:
chr10 89720744, positioned within PTEN gene;
chr7 112461939, positioned within BMT2 gene;
chr12 89985005, positioned within ATP2B1 gene;
chr17 29677227, positioned within NF1 gene;
chr11 88338063, positioned within GRM5 gene;
chr10 89624245, positioned within PTEN gene;
chr4 115544340, positioned within UGT8 gene;
chr13 47409732, positioned within HTR2A gene;
chr1 227843477, positioned within ZNF678 gene;
chr19 47424921, positioned within ARHGAP35 gene;
chr8 121228689, positioned within COL14A1 gene.

In another embodiment, the disclosed methods, systems, and components further involve testing for the presence of the change in any one or more of the following sites from Table 1:

chr18 50832017, positioned within DCC gene;
chr7 39745749, positioned within RALA gene;
chr11 60468341, positioned within MS4A8 gene;
chrX 110970087, positioned within ALG13 gene;
chr18 74635035, positioned within ZNF236 gene;
chrX 79942391, positioned within BRWD3 gene;
chr2 113417110, positioned within SLC20A1 gene;
chrX 99662008, positioned within PCDH19 gene;
chr9 5968511, positioned within KIAA2026 gene;
chrX 74519615, positioned within UPRT gene;
chr6 31779382, positioned within HSPA1L gene;
chr19 52825339, positioned within ZNF480 gene;
chr3 370022, positioned within CHL1 gene;
chr18 53017619, positioned within TCF4 gene;
chr6 101296418, positioned within ASCC3 gene;
chr2 9098719, positioned within MBOAT2 gene;
chr19 12501557, positioned within ZNF799 gene;
chr18 54281690, positioned within TXNL1 gene;
chr16 68598492, positioned within ZFP90 gene;
chr10 128908585, positioned within DOCK1 gene;
chr1 78428511, positioned within FUBP1 gene;
chrX 119678368, positioned within CUL4B gene;
chr8 53558288, positioned within RB1CC1 gene.

In a next possible embodiment, a 19-marker panel is used that covers all of the samples as listed in Table 2. In accordance with this embodiment, the disclosed methods, systems, and components involve testing for the presence of the change in the following sites from Table 1:

chr10 89720744, positioned within PTEN gene;
chr7 112461939, positioned within BMT2 gene;
chr12 89985005, positioned within ATP2B1 gene;
chr17 29677227, positioned within NF1 gene;
chr11 88338063, positioned within GRM5 gene;
chr10 89624245, positioned within PTEN gene;
chr4 115544340, positioned within UGT8 gene;
chr13 47409732, positioned within HTR2A gene;
chr1 227843477, positioned within ZNF678 gene;
chr19 47424921, positioned within ARHGAP35 gene;
chr8 121228689, positioned within COL14A1 gene;
chr18 50832017, positioned within DCC gene;
chr7 39745749, positioned within RALA gene;
chr11 60468341, positioned within MS4A8 gene;
chrX 110970087, positioned within ALG13 gene;
chr18 74635035, positioned within ZNF236 gene;
chrX 79942391, positioned within BRWD3 gene;
chr2 113417110, positioned within SLC20A1 gene;
chrX 99662008, positioned within PCDH19 gene.

In another embodiment, the disclosed methods, systems, and components involve testing for a presence of a hotspot P286R or a hotspot V411L mutation of POLE.

In a yet another embodiment, the disclosed methods, systems, and components involve testing for POLE hotspot mutation. Thus, in a possible embodiment, the disclosed methods, systems, and components involve analyzing for the presence or absence of an increased tumor mutational burden (TMB) in a sample obtained from a patient. The disclosed methods, systems, and components may involve testing said sample for a presence of a hotspot P286R or a hotspot V411L mutation of POLE and for a presence of a change of a cytosine or a guanine to any other nucleobase, in at least four of the following different genomic sites as mapped to GRC37 human genome assembly from Table 1: chr10 89720744, positioned within PTEN gene; (variant PTEN(i)), chr7 112461939, positioned within BMT2 gene; chr11 88338063, positioned within GRM5 gene, chr4 115544340, positioned within UGT8 gene, chr12 89985005, positioned within ATP2B1 gene, and chr17 29677227, positioned within NF1 gene; wherein detection of the presence of at least one of the changes in any of the genomic sites from Table 1 or of any of the hotspot POLE mutations is indicative of an increased tumor mutational burden (TMB).

In another embodiment, the disclosed methods, systems, and components may involve testing for the presence of the change in one of more of the following sites from Table 1:

chr12 89985005, positioned within ATP2B1 gene;
chr10 89624245, positioned within PTEN gene;
chr13 47409732, positioned within HTR2A gene;
chr1 227843477, positioned within ZNF678 gene;
chr19 47424921, positioned within ARHGAP35 gene;
chr8 121228689, positioned within COL14A1 gene;
chr18 50832017, positioned within DCC gene;
chr7 39745749, positioned within RALA gene;
chr11 60468341, positioned within MS4A8 gene;
chrX 110970087, positioned within ALG13 gene;
chr18 74635035, positioned within ZNF236 gene;
chrX 79942391, positioned within BRWD3 gene;
chr2 113417110, positioned within SLC20A1 gene;
chrX 99662008, positioned within PCDH19 gene.

In alternative embodiments, the disclosed methods, systems, and components involve testing for one of the two POLE hotspot mutation P286R or V411L with any of the following combinations of markers from Table 1. Respective results of the coverage are also provided:

BMT2+SLC20A1+PTEN(i)+2 POLE hotspots: 10 High, 47 Hyper (73% above), 57 (75%) above 15, and 85% UCEC.
BMT2+NF1+ATP2B1+PTEN(i)+2 POLE hotspots: 12 High 47 Hyper (76% above), 59 (78%) above 15, 89% UCEC.
NF1+BMT2+UGT8+PTEN(i)+2 POLE hotspots: 14 High 46 Hyper (77% above), 60 (79%) above 15, 85% UCEC
NF1+BMT2+GRM5+PTEN(i)+2 POLE hotspots: 12 High 47 Hyper 1 low (76% above), 59 (78%) above 15, 85% UCEC
BMT2+NF1+SLC20A1+PTEN(i)+2 POLE hotspots: 12 High 48 Hyper (77% above), 60 (79%) above 15, 87% UCEC
BMT2+ALG13+SLC20A1+PTEN(i)+2 POLE hotspots: 11 High 48 Hyper 1 med (77% above), 60 (79%) above 15, 85% UCEC
BMT2+GRM5+SLC20A1+PTEN(i)+2 POLE hotspots: 10 High 49 Hyper 1 low (76% above), 59 (78%) above 15, 85% UCEC
BMT2+BRWD3+SLC20A1+PTEN(i)+2 POLE hotspots: 12 High 48 Hyper (77% above), 60 (79%) above 15, 85% UCEC
BMT2+RB1CC1+SLC20A1+PTEN(i)+2 POLE hotspots: 12 High 48 Hyper (77% above), 60 (79%) above 15, 85% UCEC

In another embodiment, the disclosed methods, systems, and components invovle testing the sample for a presence of an additional mutation of POLE and/or for a presence of a mutation in EXO1 and/or MUTYH.

In another embodiment, the disclosed methods, systems, and components involve testing for an additional mutation in POLE wherein the additional mutation of POLE is one or more of the following: T1104M, A1967V, H144Q, S1644L, A456P, R1233, T2202M, P436R, R705W, S459F, S297F, A189T, P436R, L1235I, R1371, D213A, P135S, A456P, K777N, F367S.

Is some embodiments, the disclosed methods, systems, and components involve testing for any of these other POLE mutations comprising: T1104M, A1967V, H144Q, S1644L, A456P, R1233, T2202M, P436R, R705W, S459F, S297F, A189T, P436R, L1235I, R1371, D213A, P135S, A456P, K777N, F367S, wherein the presence of a detected mutation is indicative of an increased TMB.

In some embodiments, the disclosed methods, systems, and/or components comprise and/or utilize oligonucleotide reagents for testing a sample and identifying a nucleotide at a genomic site within the sample. Suitable oligonucleotide reagents may include primers or primer pairs for amplifying a polynucleotide sample comprising a genomic site to be tested.

In some embodiments, the oligonucleotide reagents comprise primer pairs that hybridize to polynucleotide sequences that flank a genomic site in a polynucleotide sample and which may be utilized to amplify the polynucleotide sample and prepare an amplicon comprising the genomic site (e.g., a genomic site of Table 1). Primer pairs may hybridize to polynucleotide sequences that flank a genomic site at selected flanking sites in order to prepare an amplicon comprising the genomic site and having a suitable size, such as at least about 50, 100, 150, 200, or 250 nucleotides, or a size range bounded by any of these values, such as 50-150 nucleotides. Suitable oligonucleotide reagents may comprise a set of primer pairs for amplifying multiple genomic sites of Table 1, for example, four or more primer pairs for amplifying four or more genomic sites of Table 1 in a polynucleotide sample.

In some embodiments, the oligonucleotide reagents comprise primers for sequencing a polynucleotide sample comprising a genomic site (e.g., a genomic site of Table 1). As such, a primer may hybridize to a polynucleotide sequence upstream of a genomic site such as a sequence at least about 10, 20, 30, 40, or 50 nucleotides upstream of a genomic site or within a range bounded by any of these values such as at a sequence 30-50 nucleotides upstream of a genomic site. The primer thereafter may be utilized to sequence the polynucleotide sample and determine the identify of the nucleotide at the genomic site. Suitable oligonucleotide reagents may comprise a set of primers for sequencing multiple genomic sites of Table 1, for example, four or more primers for sequencing four or more genomic sites of Table 1 in a polynucleotide sample.

In some embodiments, the oligonucleotide reagents comprise probes that hybridize to a genomic site (e.g., a genomic site of Table 1). Suitable probes may include probes that hybridize to a mutation at a genomic site and/or probes that hybridize to a wild-type sequence or control sequence at a genomic site. Alternatively, suitable probes may include probes that hybridize to a mutation at a genomic site that are possibly provided together with probes that hybridize to a wild-type sequence or control sequence at a genomic site. Suitable oligonucleotide reagents may comprise a set of probes for hybridizing to multiple genomic sites of Table 1, for example, four or more probes for hybridizing to four or more genomic sites of Table 1 in a polynucleotide sample.

In another embodiment, the disclosed methods, systems, and components involve testing the sample for a presence of one or more mutations is performed using at least one oligonucleotide specific to hybridize with said at least one or more mutations. The oligonucleotide can be a primer or a probe. As the advantage of the provided herein methods over NGS alternatives is a limited number of markers, the present methods could potentially be performed using a PCR-based assay comprising e.g. mutation-specific oligonucleotides like primers (e.g. Taqman primers) or detection probes. In another embodiment, the, the disclosed methods, systems, and components comprise oligonucleotides (e.g. primers or primers and probes) for performing a multiplex PCR. In accordance with this embodiment, such methods may be comprising performing a multiplex PCR in one or more reaction tubes or chambers, e.g. chambers of an integrated detection cartridge.

In some embodiments, the disclosed methods comprise detecting in a polynucleotide sample (e.g., a genomic DNA sample) a change of a cytosine or a guanine to any other nucleobase (likely adenine or thymine) at four or more genomic sites from Table 1 as mapped to GRC37 human genome assembly, wherein detecting comprises amplifying at least a portion of the DNA sample and sequencing the amplified portion to detect the change. In some embodiments, the disclosed methods may comprise detecting the change at the following four genomic sites: chr10 89720744, positioned within PTEN gene; chr7 112461939, positioned within BMT2 gene; chr12 89985005, positioned within ATP2B1 gene; and chr17 29677227, positioned within NF1 gene. Optionally, the method may comprise: (a) amplifying a DNA sample to prepare DNA amplicons comprising the following four genomic sites: chr10 89720744, positioned within PTEN gene; chr7 112461939, positioned within BMT2 gene; chr12 89985005, positioned within ATP2B1 gene; and chr17 29677227, positioned within NF1 gene; and (b) sequencing the DNA amplicons to detect the mutation. In further embodiments, the methods may comprise detecting for a further one or more of the changes at the sites as listed in Table 1, analogously as described above. Optionally, the DNA sample is obtained from a patient having cancer and the method further comprises administering treatment for cancer to the patient (optionally comprising administering immunotherapy to the patient and/or non-immunotherapy to the patient such as chemotherapy, radiotherapy, and/or surgery (e.g., tumor resection).

In some embodiments, the disclosed systems comprise reagents for detecting a change of a cytosine or a guanine in a DNA sample to any other nucleobase at four or more genomic sites from Table 1 as mapped to GRC37 human genome assembly, optionally wherein the reagents comprise components for amplifying at least a portion of the DNA sample and reagents for sequencing the amplified portion in order to detect the change. In further possible embodiments, the systems may comprise reagents for detecting for a further one or more of the changes at the sites as listed in Table 1, analogously as described above. In some embodiments, the reagents comprise components for amplifying at least a portion of a DNA sample comprising the following four genomic sites: chr10 89720744, positioned within PTEN gene; chr7 112461939, positioned within BMT2 gene; chr12 89985005, positioned within ATP2B1 gene; and chr17 29677227, positioned within NF1 gene; and components for sequencing the genomic site. Optionally, the system is at least partially automated and/or may comprise a hardware processor that is programmed to perform and/or to actuate a mechanical component of the system to perform one or more tasks selected from: (i) receiving and/or transporting a sample into the system; (ii) adding one or more components, reagents, and/or tools to the sample (e.g., one or more components, reagents, and/or tools to perform PCR and/or sequencing four or more of the genomic sites listed in Table 1); (iii) performing PCR on the sample; (iv) detecting a PCR product (e.g., a PCR product of four or more of the genomic sites listed in Table 1; (v) sequencing at least four or more of the genomic sites listed in Table 1; (vi) generating a report that indicates the nucleotide at four or more genomic sites listed in Table 1.

The disclosed systems and components may comprise one or more cartridges. As used herein, the term “cartridge” is to be understood as a self-contained assembly of chambers and/or channels, which is formed as a single object that can be transferred or moved as one fitting inside or outside of a larger instrument that is suitable for accepting or connecting to such cartridge. A cartridge and its instrument can be seen as forming an automated system, further referred to as an automated platform. Some parts contained in the cartridge may be firmly connected whereas others may be flexibly connected and movable with respect to other components of the cartridge. Analogously, as used herein the term “fluidic cartridge” shall be understood as a cartridge including at least one chamber or channel suitable for treating, processing, discharging, or analysing a fluid, preferably a liquid. An example of such cartridge is given in WO2007004103. Advantageously, a fluidic cartridge can be a microfluidic cartridge. In general, as used herein the terms “fluidic” or sometimes “microfluidic” refers to systems and arrangements dealing with the behaviour, control, and manipulation of fluids that are geometrically constrained to a small, typically sub-millimetre-scale in at least one or two dimensions (e.g. width and height or a channel). Such small-volume fluids are moved, mixed, separated or otherwise processed at micro scale requiring small size and low energy consumption. Microfluidic systems include structures such as micro pneumatic systems (pressure sources, liquid pumps, micro valves, etc.) and microfluidic structures for the handling of micro, nano- and picolitre volumes (microfluidic channels, etc.). Exemplary and very suitable in the present context fluidic systems were described in EP1896180, EP1904234, and EP2419705. In line with the above, the term “chamber” is to be understood as any functionally defined compartment of any geometrical shape within a fluidic or microfluidic assembly, defined by at least one wall and comprising the means necessary for performing the function which is attributed to this compartment. Along these lines, “amplification chamber” is to be understood as a compartment within a (micro)fluidic assembly, which suitable for performing and purposefully provided in said assembly in order to perform amplification of nucleic acids. Examples of an amplification chamber include a PCR chamber and a qPCR chamber. In accordance with the above, in alternative embodiments, such cartridges and/or integrated systems are provided comprising one or more oligonucleotides specific to hybridize to a sequence containing at least one of the changes of a cytosine or a guanine at four or more genomic sites from Table 1 as mapped to GRC37 human genome assembly. Optionally, the disclosed cartridges may comprise oligonucleotide primers for amplifying and/or sequencing one or more genomic sites as listed in Table 1. Such primers can be designed to flank within a reasonable upstream or downstream range of nucleotides the changes of a cytosine or a guanine at four or more genomic sites from Table 1 (exemplary ranges of nucleotides were mentioned above), or a primer can be designed to cover a change of a cytosine or a guanine from Table 1, for example if an ARMS primer approach would be desired.

In further embodiments, the disclosed methods, systems, and components involve identifying TMB-affected samples independently of their MSI-status. The disclosed methods, systems, and components may involve analyzing for the presence of microsatellite instability (MSI) in the sample.

In another embodiment, the disclosed methods, systems, and components involve assessing test samples to determining whether the test samples are microsatellite-stable. In accordance with this embodiment, the disclosed methods, systems, and components may involve determining that the sample is microsatellite stable (MSS).

In another embodiment, in view of the shifting paradigm in cancer field that focuses on pan-cancer approaches rather than limiting marker-screening methods to tumors of specific tissues of origin, the disclosed methods, systems, and components may be utilized for assessing any type of cancer sample, i.e. a cancer sample derived from any tissue type. This is in particular in line with the fact that the present methods have the potential of identifying ICB responders that cannot be identified by most commercially-available methods and because ICB is considered a pan-cancer treatment, that is not restricted to a specific cancer tissue type. In alternative embodiments, the disclosed methods, systems, and components may be utilized for assessing any tumor samples derived from tissues as listed in Table 2, and are optionally are performed on endometrial cancer samples (UCEC) and/or colorectal cancer samples (COAD).

As already mentioned throughout this description, the major advantage of the herein presented methods is that they have the promising potential of identifying responders to ICB, who could otherwise be missed by other more prevalently available methods, such as MSI-testing. Hence, in an advantageous embodiment, methods are provided further comprising the step of classifying the patient from whom the sample was obtained as a responder to immunotherapy, preferably being immunotherapy comprising treatment with an antibody specific against at least one selected from: PD-1, PD-L1, CTLA4, TIM-3, and/or, LAG3. As such, the disclosed methods may include a step of administering therapy to a patient in need thereof, such as administering immunotherapy against a target selected from PD-1, PD-L1, CTLA4, TIM-3, and/or, LAG3 (e.g., antibody therapy against PD-1, PD-L1, CTLA4, TIM-3, and/or, LAG3).

In line with the above, one can also envisage uses of the described herein methods, cartridges and systems in TMB testing and in classification of patients for immunotherapy, said therapy preferably comprising an ICB treatment, most preferably with any antibodies specific to PD-1, PD-L1, CTLA4, TIM-3, and/or, LAG3.

### EXAMPLES

1. Identification of Polymerase Epsilon (POLE) Scarring Signature in Endometrial Tumors (UCEC) from TCGA

Maintenance of DNA replication fidelity is believed to depend on a fine balance between the unique errors by polymerases δ and ε, (Korona et al., 2011, Nucl Acids Res) the equilibrium between proofreading and MMR, and distinction in nucleotide processing during the lagging and leading strand synthesis (Lujan et al., 2016, Crit Rev in Biochem and Molec Biol). Extensive studies in yeast models have shown that mutations in the exonuclease domain of Polδ and Polε homologues can cause a mutator phenotype (Skoneczna et al. 2015, FEMS Microbiol Rev).

Based on the above, in order to identify possible set of markers to detect POLE and POLD1 genes deficiency (respectively encoding for catalytic subunits of polymerases ε and δ), we decided to define a discovery data set using The Cancer Genome Atlas (TCGA) database. We chose to focus on endometrial cancer samples (UCEC), which was previously reported by The Cancer Genome Atlas Research Network (Levine et al., 2013, Nature) to relatively frequently carry POLE and POLD1 mutations. At the time of the analysis, TCGA contained 524 UCEC samples in total. Based on the microsatellite instability (MSI) annotations provided by TCGA, 165 of the samples were MSI-positive (annotated as MSI-L or MSI-H, i.e. having low MSI or high MSI) samples. For our discovery, we only focused on the remaining 359 microsatellite stable (annotated as MSS) TCGA-UCEC samples, due to the fact there currently exist efficient methods to detect MSI-positive samples and because it is believed that MSI-positive tumors share different characteristics than MSS POLE-deficient tumors.

Among the 359 TCGA-UCEC-MSS samples, we identified 32 samples with one of the two POLE hotspot mutations (P286R and V411L), 13 samples with other POLE mutations and 12 samples with POLD1 mutations. 9 out of the 12 samples with POLD1 mutations also contained POLE mutations. We then plotted the Tumor Mutational Burden (TMB) values defined as number of somatic (tumor vs matched normal sample, WES variant calling, comprising both synonymous and non-synonymous mutations, but not including indels) substitutions per coding Mb. The results are shown in FIG. 1 for the following sample groups: MSI-positive UCEC samples (“MSI”, including both MSI-L and MSI-H), MSS UCEC samples with POLE P286R or V411L mutation (“POLE hotspot”), MSS UCEC samples with POLE-non-hostspot mutations (“POLE others”), MSS UCEC samples with a POLD1 mutation (“POLD1”), and MSS UCEC samples without a mutation in either POLE or POLD1.

As can be seen in FIG. 1, the 3 POLD1-mutated POLE-non-mutated samples (marked inside of an added circle) had a similar TMB to the samples without any POLE or POLD1 mutations, which indicates that POLD1 mutation alone does not cause hypermutator phenotype. Consequently, the rest of the marker analysis was performed using the 32 UCEC-MSS samples harboring a POLE hotspot mutation.

In order to detect recurrent marker variants, we downloaded somatic variant lists from exome-sequencing of the 32 TCGA-UCEC-MSS samples with POLE hotspot mutations. For all these the variants, we preformed the following analysis steps to detect the recurrent ones. First (1), we pooled all the variants from the 32 samples. Then (2), we excluded variants present also in any of the 314 non-POLE mutated samples. Next (3), we excluded the known variants in public databases including the 1000 Genome database (v.2015 August), dbsnp (v.138), Kaviar database (v.20150923), and hrcr1 database (first release). Then (4), we annotated the nonsynonymous/stop gain exonic mutations, and lastly (5), we selected the recurrent variants occurring in more than 6 out of 32 samples (frequency >0.18).

The result was an identification of 34 recurrent variant markers as listed in Table 3

TABLE 3

| SEQ | | | | |
|---|---|---|---|---|
| ID | position in | | | fre- |
| NO. | GRCh37/hg19 | gene name | mutation type | quency |
| 1 | chr19 47424921 | ARHGAP35 | stopgain | 0.28125 |
| 2 | chr17 29677227 | NF1 | stopgain | 0.28125 |
| 3 | chrX 99662008 | PCDH19 | nonsynonymous SNV | 0.25 |
| 4 | chr9 5968511 | KIAA2026 | nonsynonymous SNV | 0.25 |
| 5 | chr7 112461939 | BMT2 | stopgain | 0.25 |
| 6 | chrX 74519615 | UPRT | nonsynonymous SNV | 0.21875 |
| 7 | chr8 121228689 | COL14A1 | nonsynonymous SNV | 0.21875 |
| 8 | chr6 31779382 | HSPA1L | nonsynonymous SNV | 0.21875 |
| 9 | chr19 52825339 | ZNF480 | nonsynonymous SNV | 0.21875 |
| 10 | chr13 47409732 | HTR2A | nonsynonymous SNV | 0.21875 |
| 11 | chr11 60468341 | MS4A8 | nonsynonymous SNV | 0.21875 |
| 12 | chrX 110970087 | ALG13 | stopgain | 0.21875 |
| 13 | chr3 370022 | CHL1 | stopgain | 0.21875 |
| 14 | chr18 53017619 | TCF4 | stopgain | 0.21875 |
| 15 | chr6 101296418 | ASCC3 | nonsynonymous SNV | 0.1875 |
| 16 | chr4 115544340 | UGT8 | nonsynonymous SNV | 0.1875 |
| 17 | chr2 9098719 | MBOAT2 | nonsynonymous SNV | 0.1875 |
| 18 | chr19 12501557 | ZNF799 | nonsynonymous SNV | 0.1875 |
| 19 | chr18 74635035 | ZNF236 | nonsynonymous SNV | 0.1875 |
| 20 | chr18 54281690 | TXNL1 | nonsynonymous SNV | 0.1875 |
| 21 | chr16 68598492 | ZFP90 | nonsynonymous SNV | 0.1875 |
| 22 | chr12 89985005 | ATP2B1 | nonsynonymous SNV | 0.1875 |
| 23 | chr11 88338063 | GRM5 | nonsynonymous SNV | 0.1875 |
| 24 | chr10 128908585 | DOCK1 | nonsynonymous SNV | 0.1875 |
| 25 | chr1 78428511 | FUBP1 | nonsynonymous SNV | 0.1875 |
| 26 | chr1 227843477 | ZNF678 | nonsynonymous SNV | 0.1875 |
| 27 | chrX 79942391 | BRWD3 | stopgain | 0.1875 |
| 28 | chrX 119678368 | CUL4B | stopgain | 0.1875 |
| 29 | chr8 53558288 | RB1CC1 | stopgain | 0.1875 |
| 30 | chr7 39745749 | RALA | stopgain | 0.1875 |
| 31 | chr2 113417110 | SLC20A1 | stopgain | 0.1875 |
| 32 | chr18 50832017 | DCC | stopgain | 0.1875 |
| 33 | chr10 89720744 | PTEN | stopgain | 0.1875 |
| | | (“PTEN(i)”) | | |
| 34 | chr10 89624245 | PTEN | stopgain | 0.1875 |
| | | (“PTEN(ii)”) | | |

For the 40 detected POLE deficient TCGA-UCEC-MSS samples (including 32 with hotspot mutations and 8 with other mutations), using Pearson correlation we correlated the number of scored positive markers and TMB level/sample, the result of which is shown in FIG. 2. The correlation coefficient was 0.31 indicating that the correlation is insignificant. Despite no correlation being found, the results of the experiment are interesting as they remarkably indicate that every single mutation of the identified set on its own is specifically associated with an increased TMB.

2. Search for POLE Scarring Signature in Colorectal Tumors (COAD) from TCGA and Additional Other MSS-POLE-Hotspot Tumors from TCGA

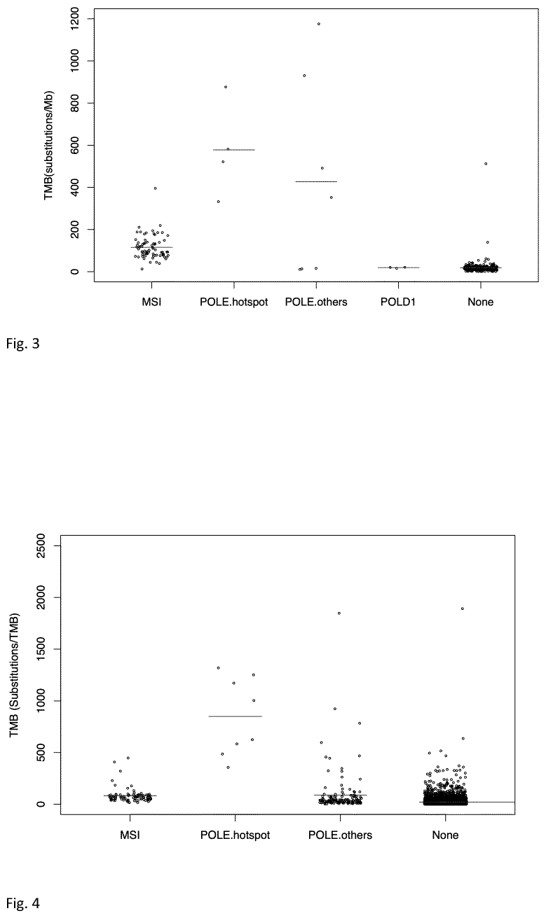

Secondly, we performed the same analysis using colorectal 428 colorectal samples (COAD) from TCGA available in TCGA. Among these samples, 72 samples were annotated as MSI-H and 356 samples were annotated as MSS. Out of the 356 TCGA-COAD-MSS samples, 4 samples contained a POLE hotspot mutation, 7 samples contained at least one other POLE mutation (non-hotspot), and 3 samples had POLD1 mutations. We then plotted the TMB levels in different categories of samples as it was done for UCEC samples, as described above. The results are shown in FIG. 3.

As in the UCEC MSS sample analysis, the POLD1-mutated POLE-non-mutated COAD samples did not show elevated TMB, confirming the previous observation that POLD1 mutation alone does not cause hypermutator phenotype.

The recurrent variant search was performed as described above, using the 4 identified TCGA-COAD-MSS samples harboring a POLE hotspot mutation. No recurrent mutations were found in these samples, which can be attributed to the very low number of samples used for the analysis.

Not having identified recurrent variants in TCGA-COAD-MSS-POLE-hotspot samples, we then searched among all the other cancer types in TCGA database (i.e. not TCGA-UCEC and TCGA-COAD) for other MSS tumor samples harboring a POLE-hotspot mutation. We found that TCGA listed 8 of them as shown in the “POLE hotspot” group of FIG. 4. Among them, 4 samples carried the P286R hotspot mutation and included the following: 1 sample from Rectal cancer (READ), 1 from Pancreatic cancer (PAAD), 1 from Bladder cancer (BLCA) and 1 from Breast cancer (BRCA). The remaining 4 carried the V411L hotspot and included the following: 1 READ, 1 stomach cancer (STAD), 1 Glioblastoma (GBM), and 1 Cervical cancer (CESC). Additionally, TCGA contained 140 MSS non-UCEC and non-COAD cancer samples with other POLE mutations not being hotspots, shown in the “POLE-others” group in FIG. 4, several of which had elevated TMB.

To all the above mentioned 8 TCGA-non-UCEC and TCGA-non-COAD MSS POLE-hotspot samples, we then applied the discovery approach as described above but also could not identify any recurrent variants.

### 3. Retrospective Application of the POLE-Mutation Signature Marker Panel as Identified in UCEC-POLE-Hotspot-Mutated Samples Over all UCEC TCGA Records

In view of the lack of recurrent variants in COAD or other cancer non-UCEC samples, we defined the 34 recurrent mutations as identified in UCEC tumors as the initial 34-POLE-mutation-signature marker panel for detecting POLE-deficient tumors in TCGA records.

We first applied the initial 34-marker panel to all 524 TCGA-UCEC samples to estimate its sensitivity and specificity. For each sample, we overlapped 34-marker-panel with its variant list and checked how many variants out of the 34 potential markers can be detected per sample. If one variant (i.e. one marker) is detected in a certain sample, we consider that the sample is positive for this variant.

As a result, we detected 47 TCGA-UCEC samples having at least one positive marker. We defined these samples as POLE-deficient samples. The 47 detected POLE-deficient samples included: (i) all 32 samples with POLE hotspot mutations used to define the initial 34-marker-panel, (ii) 1 MSI-H sample with POLE hotspot mutation, (iii) 6 MSI-H samples with other POLE mutations, (iv) 8 MSS samples with other POLE mutations. Since we were not interested in MSI-H samples in this analysis, we further investigated the 8 MSS samples with other POLE mutations. Details about the samples are provided in the Table 4 below (wherein “MSS”=microsatellite stable; “MSI-L” or “MSI-H”=MSI positive; “Hotspot”—POLE hotspot mutation present; “POLE”=POLE non-hotspot mutation present; “EXO1”=EXO1 mutation present; “MUTYH”=MUTYH mutation present, “NA”=presence of the mutation of interest not indicated in TCGA; TMB expressed as substitutions/Mb, not containing indels).

TABLE 4

| PatientID | Cancer | nrPos | TMB | MSI | POLE | EXO1 | MUTYH |
|---|---|---|---|---|---|---|---|
| TCGA-A5-A0G2 | UCEC | 6 | 3217.9 | MSI-L | Hotspot | NA | NA |
| TCGA-AP-A0LM | UCEC | 10 | 1826.3 | MSS | Hotspot | NA | NA |
| TCGA-AX-A2HC | UCEC | 1 | 1788.8 | MSI-H | POLE | NA | NA |
| TCGA-EO-A3B0 | UCEC | 12 | 1723.6 | MSS | Hotspot | NA | NA |
| TCGA-EO-A22R | UCEC | 2 | 1669.4 | MSI-L | Hotspot | NA | NA |
| TCGA-E6-A1LX | UCEC | 8 | 1651.6 | MSI-L | Hotspot | NA | NA |
| TCGA-FI-A2D5 | UCEC | 2 | 1603.8 | MSS | Hotspot | NA | NA |
| TCGA-EO-A22U | UCEC | 7 | 1564.1 | MSI-H | Hotspot | NA | NA |
| TCGA-AP-A1DV | UCEC | 1 | 1478.9 | MSI-L | POLE | NA | NA |
| TCGA-B5-A3FA | UCEC | 3 | 1360.5 | MSI-L | Hotspot | NA | NA |
| TCGA-EO-A22X | UCEC | 11 | 1359.7 | MSS | Hotspot | NA | NA |
| TCGA-BS-A0UF | UCEC | 9 | 1346.7 | MSS | Hotspot | NA | NA |
| TCGA-AX-A1CE | UCEC | 1 | 1310.2 | MSI-H | POLE | NA | NA |
| TCGA-A5-A0G1 | UCEC | 1 | 1304.2 | MSI-H | POLE | NA | NA |
| TCGA-B5-A0JY | UCEC | 12 | 1289.8 | MSS | Hotspot | NA | NA |
| TCGA-A5-A2K5 | UCEC | 8 | 1273.5 | MSS | Hotspot | NA | NA |
| TCGA-AP-A056 | UCEC | 13 | 1255.3 | MSI-L | Hotspot | NA | NA |
| TCGA-B5-A11E | UCEC | 4 | 1238.9 | MSI-L | Hotspot | NA | NA |
| TCGA-BS-A0UV | UCEC | 5 | 1236.7 | MSI-L | Hotspot | NA | NA |
| TCGA-AX-A05Z | UCEC | 12 | 1188.8 | MSS | Hotspot | NA | NA |
| TCGA-DF-A2KU | UCEC | 3 | 1154.1 | MSI-L | Hotspot | NA | NA |
| TCGA-AJ-A3EL | UCEC | 13 | 1116.2 | MSI-L | Hotspot | NA | NA |
| TCGA-AX-A0J0 | UCEC | 15 | 1091.8 | MSI-L | Hotspot | NA | NA |
| TCGA-AX-A06F | UCEC | 1 | 994.8 | MSI-L | POLE | NA | NA |
| TCGA-AP-A051 | UCEC | 1 | 985.2 | MSI-H | POLE | NA | NA |
| TCGA-D1-A103 | UCEC | 2 | 974.9 | MSI-L | POLE | NA | NA |
| TCGA-B5-A1MR | UCEC | 1 | 852.7 | MSS | POLE | NA | NA |
| TCGA-D1-A17Q | UCEC | 6 | 760.3 | MSI-L | Hotspot | NA | NA |
| TCGA-AJ-A3EK | UCEC | 2 | 735.6 | MSI-H | POLE | NA | NA |
| TCGA-EO-A3AV | UCEC | 11 | 634.5 | MSS | Hotspot | NA | NA |
| TCGA-AP-A1E0 | UCEC | 4 | 629.4 | MSI-L | POLE | NA | NA |
| TCGA-EY-A1GI | UCEC | 9 | 613.5 | MSS | Hotspot | NA | NA |
| TCGA-BK-A6W3 | UCEC | 3 | 609.7 | MSS | Hotspot | NA | NA |
| TCGA-EO-A3AY | UCEC | 8 | 590.8 | MSS | Hotspot | NA | NA |
| TCGA-AJ-A5DW | UCEC | 4 | 476.8 | MSS | Hotspot | NA | NA |
| TCGA-EY-A1G8 | UCEC | 5 | 358.6 | MSS | Hotspot | NA | NA |
| TCGA-D1-A16X | UCEC | 9 | 354 | MSS | Hotspot | NA | NA |
| TCGA-E6-A1M0 | UCEC | 2 | 272.9 | MSS | POLE | NA | NA |
| TCGA-AJ-A3BH | UCEC | 1 | 260.2 | MSI-H | POLE | NA | NA |
| TCGA-A5-A0GP | UCEC | 1 | 247.6 | MSS | Hotspot | NA | NA |
| TCGA-B5-A11N | UCEC | 3 | 240.7 | MSI-L | Hotspot | NA | NA |
| TCGA-QF-A5YS | UCEC | 6 | 236.3 | MSS | Hotspot | NA | NA |
| TCGA-BS-A0TC | UCEC | 1 | 217.5 | MSS | POLE | NA | NA |
| TCGA-DF-A2KV | UCEC | 1 | 188.4 | MSS | POLE | NA | NA |
| TCGA-EY-A1GD | UCEC | 3 | 159.7 | MSS | Hotspot | NA | NA |
| TCGA-D1-A16Y | UCEC | 3 | 145.8 | MSI-L | Hotspot | NA | NA |
| TCGA-QS-A5YQ | UCEC | 3 | 132.4 | MSS | Hotspot | NA | NA |

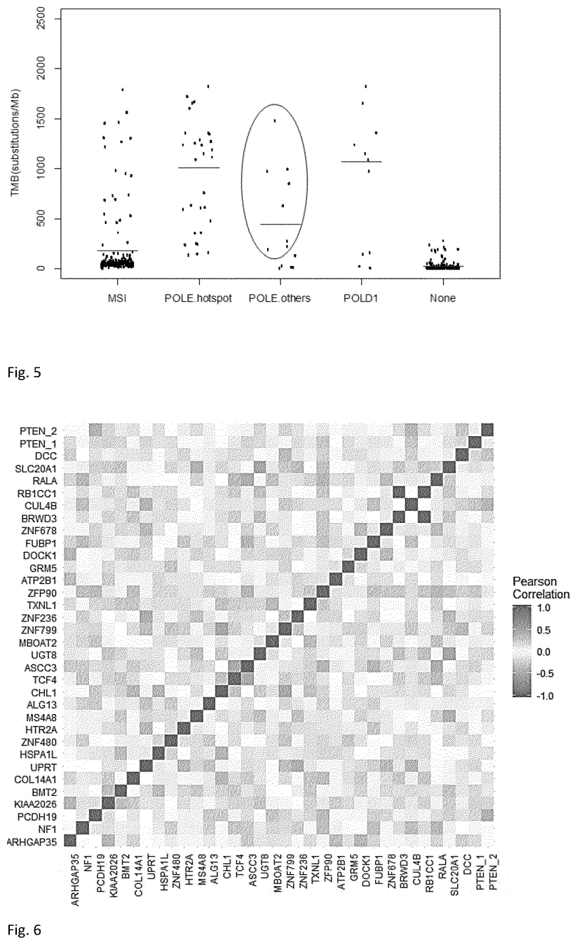

As further shown in FIG. 5, the identified 8 UCEC MSS samples (encircled in the FIG. 5) had all elevated TMB, the minimal TMB observed being 188.4 substitutions/Mb. More details about these samples, listing the exact POLE non-hotspot mutations found in them, are provided in Table 5 below. Of note, the above mentioned lowest TMB of 188.4 substitutions/Mb as observed in the sample TCGA-DF-A2 KV is even higher than the TMB observed in the POLE-hotspot-containing MSS samples TCGA-EY-A1GD, and TCGA-QS-A5YQ (cf Table 4 above), which strongly suggests that the herein listed POLE non-hotspot mutations can effectively disable the proper function of the polymerase E.

TABLE 5

| | | | No. | | | |
|---|---|---|---|---|---|---|
| | | | positive | | MSI | |
| # | Patient ID | Cancer | markers | TMB | status | POLE non-hotspot mutations |
| 1 | TCGA-AP-A1E0 | UCEC | 4 | 629.4 | MSI-L | S459F |
| 2 | TCGA-D1-A103 | UCEC | 2 | 974.9 | MSI-L | T1104M; A1967V; H144Q; S1644L; A456P |
| 3 | TCGA-E6-A1M0 | UCEC | 2 | 272.9 | MSS | S459F |
| 4 | TCGA-BS-A0TC | UCEC | 1 | 217.5 | MSS | M444K |
| 5 | TCGA-DF-A2KV | UCEC | 1 | 188.4 | MSS | A456P |
| 6 | TCGA-B5-A1MR | UCEC | 1 | 852.7 | MSS | R750W |
| 7 | TCGA-AX-A06F | UCEC | 1 | 994.8 | MSI-L | R1233*; T2202M; P436R |
| 8 | TCGA-AP-A1DV | UCEC | 1 | 1478.9 | MSI-L | S297F |

The above results show that the initial 34-marker-panel is capable of detecting not only the discovery set of UCEC samples with POLE hotspot mutations, but also other POLE-deficient samples with substantially elevated TMB of at least above 188.4 substitutions/MB. The above is further supported by Table 6, which shows the amount of MSS UCEC samples detected by the 34-marker panel (i.e. if at least 1 variant is detected) out of all MSS-UCEC samples in TCGA per different TMB level ranges.

TABLE 6

| | all MSS | panel detected |
|---|---|---|
| TMB | UCEC samples | samples |
| ranges | (n = 395) | (n = 40) |
| 0-10 | 111 | 0 |
| 0-50 | 187 | 0 |
| 50-100 | 8 | 0 |
| 100-200 | 14 | 4 |
| 200-300 | 8 | 5 |
| >300 | 31 | 31 |

### 4. Application of the POLE-Mutation Signature Marker Panel as Identified in UCEC-POLE-Hotspot-Mutated Samples Over all Cancer Types in TCGA Records Excluding UCEC Samples

We then applied the 34-marker-panel to all 7346 TCGA sample records, including both the MSI-positive and MSS samples, which belong to 14 different cancer types excluding the TCGA-UCEC samples analyzed above. We screened the variant lists of all the samples using the initial 34-marker-panel in order to test how many positive markers can be identified per sample. If a sample contained at least one (>0) positive maker, we considered it as comprising a signature proper to POLE-deficient samples.

In total, we identified 35 samples across 10 different cancer types with said POLE-deficiency-signature. In these 35 samples, 3 samples were MSI-H, 11 samples contained one of the POLE hotspot mutations, 8 samples contained at least one other POLE mutation (1 out of the 8 being an MSI-H sample, the remaining 7 being MSS samples with high TMB ranging from 262.1 to 1846.8 substitutions/MB); 6 samples had an EXO1 somatic mutation (2 out of the 6 being EXO1 mutated but not POLE-mutated), and, lastly, 4 samples had MUYTH somatic mutations (all of which notably having also POLE mutations, 3 containing a POLE hotspot mutation). Detailed information about the detected samples is provided in the Table 7 (wherein “MSS”=microsatellite stable; “MSI-L” or “MSI-H”=MSI positive; “Hotspot”—POLE hotspot mutation present; “POLE”=POLE non-hotspot mutation present; “EXO1”=EXO1 mutation present; “MUTYH”=MUTYH mutation present, “NA”=presence of the mutation of interest not indicated in TCGA; TMB expressed as substitutions/Mb, not containing indels).

TABLE 7

| PatientID | Cancer | nrPos | TMB | MSI | POLE | EXO1 | MUTYH |
|---|---|---|---|---|---|---|---|
| TCGA-FW-A3R5 | SKCM | 3 | 1891.5 | MSS | NA | EXO1 | NA |
| TCGA-AG-A002 | READ | 7 | 1846.8 | MSS | POLE | NA | MUTYH |
| TCGA-IB-7651 | PAAD | 1 | 1318.3 | MSS | Hotspot | EXO1 | NA |
| TCGA-06-5416 | GBM | 2 | 1171.3 | MSS | Hotspot | EXO1 | NA |
| TCGA-F5-6814 | READ | 12 | 1002.7 | MSS | Hotspot | EXO1 | NA |
| TCGA-CA-6717 | COAD | 3 | 930.4 | MSS | POLE | EXO1 | NA |
| TCGA-AZ-4315 | COAD | 3 | 876.5 | MSS | Hotspot | NA | MUTYH |
| TCGA-AA-A00N | COAD | 2 | 797.5 | MSI-L | Hotspot | NA | NA |
| TCGA-AN-A046 | BRCA | 7 | 623.8 | MSS* | Hotspot | NA | NA |
| TCGA-19-5956 | GBM | 3 | 596.1 | MSS | POLE | NA | NA |
| TCGA-BR-8680 | STAD | 1 | 582.8 | MSS | Hotspot | NA | NA |
| TCGA-AA-3984 | COAD | 4 | 581.3 | MSS | Hotspot | NA | NA |
| TCGA-EI-6917 | READ | 6 | 484.5 | MSS | Hotspot | NA | MUTYH |
| TCGA-VQ-A8P2 | STAD | 2 | 447 | MSI-H | POLE | NA | NA |
| TCGA-AA-3977 | COAD | 3 | 361.9 | MSS | POLE | NA | NA |
| TCGA-DK-A6AW | BLCA | 3 | 355.9 | MSS | Hotspot | NA | MUTYH |
| TCGA-AA-3510 | COAD | 3 | 352.1 | MSS | POLE | NA | NA |
| TCGA-CA-6718 | COAD | 1 | 332 | MSS | Hotspot | NA | NA |
| TCGA-AG-3892 | READ | 4 | 317.5 | MSS | POLE | NA | NA |
| TCGA-FR-A8YC | SKCM | 1 | 276 | MSS | NA | EXO1 | NA |
| TCGA-FU-A3HZ | CESC | 2 | 262.1 | MSS | POLE | NA | NA |
| TCGA-XN-A8T3 | PAAD | 6 | 173 | MSS | NA | NA | NA |
| TCGA-D3-A5GO | SKCM | 1 | 144.9 | MSS | NA | NA | NA |
| TCGA-WE-A8K5 | SKCM | 1 | 107 | MSS | NA | NA | NA |
| TCGA-D3-A51G | SKCM | 1 | 106.5 | MSS | NA | NA | NA |
| TCGA-FR-A3YO | SKCM | 1 | 79.1 | MSS | NA | NA | NA |
| TCGA-FS-A4F2 | SKCM | 1 | 75.2 | MSS | NA | NA | NA |
| TCGA-YB-A89D | PAAD | 2 | 56.4 | MSS | NA | NA | NA |
| TCGA-VQ-A8PB | STAD | 1 | 44.4 | MSI-H | NA | NA | NA |
| TCGA-VQ-A91E | STAD | 1 | 43.9 | MSI-H | NA | NA | NA |
| TCGA-DM-A28C | COAD | 1 | 15.4 | MSS | NA | NA | NA |
| TCGA-33-AASJ | LUSC | 1 | 13.3 | MSS | NA | NA | NA |
| TCGA-IN-A6RP | STAD | 1 | 11.6 | MSS | NA | NA | NA |
| TCGA-41-3392 | GBM | 1 | 5.3 | MSS | NA | NA | NA |
| TCGA-VQ-A8PD | STAD | 1 | 4.9 | MSS | NA | NA | NA |

From the above Table 7, it can also be seen that the 34 panel identified 12 non-UCEC tumor samples (marked in bold) with a TMB lower than the lowest TMB observed in among MSS UCEC POLE-hotspot containing samples used for constructing the discovery panel. (i.e. sample TCGA-QS-A5YQTMB=132.4 subs/Mb; cf Table 4). 2 of these samples were MSI-H (Stomach Adenocarcinoma or STAD samples TCGA-VQ-A8PB and TCGA-VQ-A91E), which can explain the low assigned to them TMB value as the values presented here do not include indels. The remaining 10 samples are annotated MSS and based on the TCGA records do not contain mutations in any of POLE, EXO1, MUTYH, and with the exception of melanomas (i.e. SKCM samples TCGA-WE-A8K5, TCGA-D3-A51G, TCGA-FR-A3YO and TCGA-FS-A4F2) are derived from primary, i.e. possibly early stage, tumors. Despite low TMB values and lack of key driver mutations, we still believe the detection of these samples by the 34 panel is valuable and may hint towards a good ICB responder status. Especially that, as we explained above, TMB values are highly unreliable on their own and differ depending on the test used. For example, findings in SCLC, NSCLS, and urothelial carcinoma show that TMB thresholds for selecting good responders for ICB correspond to ≥10 mutations per megabase (mut/Mb) by Foundation One testing or to ≥7 mut/Mb by MSK-IM PACT testing (Antonia et al., 2017, World Conf on Lung Canc; Abstract OA 07.03a; Kowanetz rt al., 2016, Ann Oncol; Powles et al., 2018, Genitourinary Canc Symp); and that by applying higher thresholds of equal to 16.2 mut/Mb (Kowanetz et al., J Thoracic Oncol) or 15 mut/Mb (Ramalingam et al., 2018, AACR Ann Meeting, Abstract #1137) did not increase the efficacy for different treatments. Consequently, we hypothesize that these samples could potentially sill be derived from good responders, only that their tumors were still at the early stage or had other DNA surveillance mechanisms affected than the ones related to POLE deficiency. The latter may be further supported by the fact that more than ⅓ of these MSS samples are melanomas (SKCM samples), where the mutation-acquirement mechanism is known to be driven by UV damage, and which do not need to have highly elevated TMB to generate immuno-reactive neoantigens (Gubin et al., 2014, Nature).

Then, as shown in FIGS. 4 and 5, by the application of the proposed herein initial panel, we notably also identified in the TCGA database 12 non-UCEC MSS samples containing a POLE-hotspot mutation. In detail, they contained the 4 MSS POLE-hotspot COAD samples shown in FIG. 3 and the 8 MSS POLE hotspot non-COAD/non-UCEC samples shown in FIG. 4. Then, we confirmed that 11 out of these 12 samples were positive for at least one of the 34-insitial signature marker panel. The 12^{th} sample could not be confirmed, likely due to incomplete TCGA annotation.

Of further note, the 7 MSS non-UCEC samples containing a POLE-non-hotspot mutation, which were pulled out from all of the TCGA records by the application of the initial POLE-scarring signature panel of the 34 markers that we identified, all had very elevated TMB, namely ranging from 262.1 to 1846.8 substitutions/MB.

This finding is in line with the result obtained from applying the 34 marker panel to all TCGA-UCEC samples, where the pulled out TCGW-UCEC-MSS samples containing a POLE other than a hotspot mutation also had a substantially elevated TMB, ranging from minimum 188.4 substitutions/MB to 1478.9 substitutions/MB.

The above results show that the initial 34 markers for identifying the POLE-dependent scarring are highly sensitive to samples carrying a POLE-mutation, being either a POLE hotspot mutation or another POLE mutation affecting the enzyme's proper function, all of which have highly elevated tumor mutational burden.

The POLE non-hotspot mutations picked in the MSS samples by the identified herein initial 34-marker panel are shown in the Table 8 below (showing TCGA-non-UCEC samples) and in the Table 5 presented above (showing TCGA-UCEC samples).

TABLE 8

| | | | No. | | | |
|---|---|---|---|---|---|---|
| | | | positive | | MSI | |
| # | Patient ID | Cancer | markers | TMB | status | POLE non-hotspot mutations |
| 1 | TCGA-AG-A002 | READ | 7 | 1846.8 | MSS | S459F |
| 2 | TCGA-AG-3892 | READ | 4 | 317.5 | MSS | S459F |
| 3 | TCGA-CA-6717 | COAD | 3 | 930.4 | MSS | L1235I; R1371* |
| 4 | TCGA-19-5956 | GBM | 3 | 596.1 | MSS | R1826W; A456P |
| 5 | TCGA-AA-3977 | COAD | 3 | 361.9 | MSS | K777N; F367S |
| 6 | TCGA-AA-3510 | COAD | 3 | 352.1 | MSS | D213A; P135S; A456P |
| 7 | TCGA-FU-A3HZ | CESC | 2 | 262.1 | MSS | F1849F; S297F |

When comparing the POLE-non-hotspot mutations as listed in the Tables 8 and 5, it can be noticed that several of these mutations are reoccurring among different samples and cancer types. For example, 4 samples (2 READ, 2 UCEC) are carrying POLE S459F mutation, 4 samples (1 GBM, 1 COAD, 2 UCEC) are carrying A456P mutation, and 2 samples show S297F mutation (1 CESC and 1 UCEC). This could be an indication of functional relevance of these and other above-listed POLE non-hotspot mutations and their causative involvement in the increased TMB phenotype.

The records shown in the Tables 8 and 5 suggest that the initially identified 34-marker-panel can be used to identify POLE-functionally deficient or impaired samples having largely increased TMB. To further support this statement, we have put together the data on COAD, PAAD, STAD, READ MSS samples that have reliably indicated MSI-status in the TCGA, and compared the numbers of these samples as detected by the 34-marker panel (i.e. having at least 1 variant detected) per different TMB level category. The data for COAD, PAAD, STAD, READ MSS samples are shown in Table 9 and the data for these and UCEC samples together are shown in Table 10 below.

TABLE 9

| | COAD, PAAD, STAD, | panel detected |
|---|---|---|
| TMB | READ MSS samples | samples |
| threshold | (n = 1009) | (n = 18) |
| 0-10 | 465 | 1 |
| 0-50 | 519 | 2 |
| 50-100 | 8 | 1 |
| 100-200 | 2 | 1 |
| 200-300 | 0 | 0 |
| >300 | 15 | 13 |

TABLE 10

| | UCEC, COAD, PAAD, | |
|---|---|---|
| | STAD, READ MSS | panel detected |
| TMB | samples together | samples |
| threshold | (n = 1368) | (n = 18) |
| 0-10 | 576 | 1 |
| 0-50 | 706 | 2 |
| 50-100 | 16 | 1 |
| 100-200 | 16 | 5 |
| 200-300 | 8 | 5 |
| >300 | 46 | 44 |

5. Further Analysis of the Strength and Redundancy of Individual Markers with the Initially-Identified 34-Marker Panel

An in depth computational analysis was initiated in order to investigate which markers showed the strongest performance in recovering samples with elevated TMB levels. To this end, all combinations of markers were exhaustively screened for their combined performance. The best performing combinations were withheld. At the same time the identification of markers displaying great levels of redundancy were identified through calculation of the co-occurrence of biomarkers. The co-occurrence between markers is shown in FIG. 6. It shows that the markers in the genes RB1CC1 and BRWD3 have a co-occurrence of 1. The other strongly correlated markers are shown in Table 11.

TABLE 11

| | | |
|---|---|---|
| ASCC3 | FUBP1 | 0.36 |
| CHL1 | HSPA1L | 0.36 |
| CUL4B | PTEN_2 | 0.36 |
| RALA | ZFP90 | 0.36 |
| ZFP90 | ASCC3 | 0.36 |
| CUL4B | UPRT | 0.37 |
| PTEN_2 | PCDH19 | 0.38 |
| SLC20A1 | MS4A8 | 0.39 |
| RALA | ASCC3 | 0.41 |
| ZNF236 | UPRT | 0.41 |
| TCF4 | ASCC3 | 0.45 |
| UGT8 | SLC20A1 | 0.46 |
| ZNF799 | CHL1 | 0.46 |

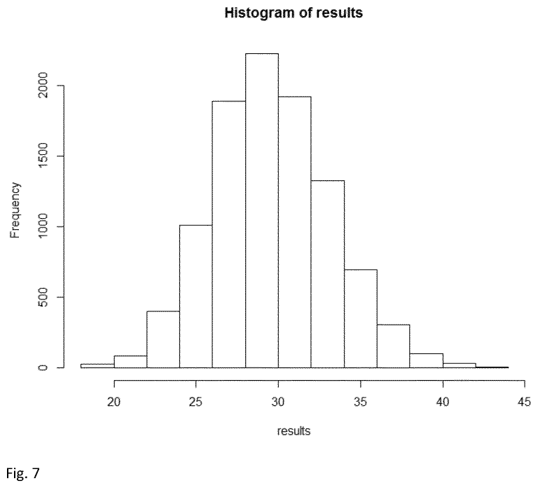

This allowed us to create a minimal experimental panel of 19 markers that covers all the samples. The number of markers per panel that was sub sampled was further reduced, to retrieve the minimal panel that could still retrieve a better sample set than could be obtained through random sampling of the markers. For performing the random sampling, we tested 10,000 randomly selected subsets of markers and evaluated their ability to retrieve samples in the dataset. The results are displayed in FIG. 7. They show that for a four marker panel, the maximum number of samples observed was 43 one time, while the median was 30. We then selected incremental in size panels of best performing biomarkers within the panel of 19 markers, starting from a minimal panel of 4 markers. The best performing panels we identified were discussed in the Detailed Description section above. We found two best performing panels of 4 markers, both including the markers in the PTEN(i), BMT2, and ATPB1 genes and an additional one in either NF1 or GRM5, which retrieve 43 or 44 samples of the 82 identified depending on inclusion of GRM5. The results of the sampling simulation illustrate that even with this minimal subset of biomarkers, an equally good score is very rarely obtained (1/10000) through random sampling, which highlights the predictive nature of the computed here minimal panels of 4 biomarkers and more for picking up samples with elevated TMB.

Next to establishing a panel based on the biomarkers, minimal panels were also created based on the biomarkers and prevalence of POLE hotspot mutations in the same manner as described above. The results of these computations were also discussed in the Detailed Description section earlier.

6. Experimental Testing of Samples with Endometrium Cancer

In an additional experiment, a series of tumor samples from patients with endometrium cancer were analyzed for the presence of an increased tumor mutational burden (TMB), using the method comprising sequencing of the different genomic sites as mapped to GRC37 human genome assembly in Table 1 for a presence of at least one mutation. The results were compared with the total number of mutations present in the regions sequenced, including the number of nucleotide variants found in a standard somatic cancer panel used in routine clinical sequencing panel consisting of a panel of 75 amplicons covering the hotspot regions of 21 of the most common cancers genes, plus an additional 25 MSI markers.

To this end, 36 formalin-fixed paraffin-embedded endometrium cancer samples were sequenced by means of 34 amplicons covering the 34 variations of Table 1. DNA was extracted from the samples by means of DNA was extracted from pathologically annotated neoplastic region(s) of the tumors using an Invitrogen PureLink™ Genomic DNA Mini Kit according to manufacturer's instructions (Invitrogen™ K182002). Targeted sequencing was performed using a custom panel (total of 134 amplicons) using an Ion PGM™ System for Next-Generation Sequencing, and analysis was performed using Torrent Suite Software for Sequencing and Data Analysis (ThermoFisher Scientific) according to manufacturer's instructions. The results are shown in Table 12. In this randomly chosen series of endometrium cancers, 10/36 (27.8%; samples 1, 2, 3, 5, 6, 7, 15, 17, 18, and 34) were positive for at least one marker. The geomean number of nucleotide variants detected in the sequencing runs was 216 for the samples containing one or more of the Table 1 markers versus a geomean of 32 variants for the samples where no variant was detected. The group containing any of the markers had an average elevated TMB of 6.75-fold compared to the control group. This confirms that this signature captures elevated TMB.

As further shown in Table 12, samples 2, 3, 6, 17, 18, and 34 contained between 2 and 7 markers. As explained above, the chance that 2 or more markers from any randomly chosen set of 34 markers would occur in a genome is virtually non-existent. Therefore, this provides further proof in an independent, real life sample set, that the markers are connected to a DNA repair failure mechanism and may be part of a resulting scarring signature in certain cancers. The samples where one marker was detected (samples 1, 5, 7, and 15) showed an geomean number of variants of 166, while those with 2 or more markers showed a geomean of 257, however, also samples with just one of the markers positive showed a clearly elevated number of variants compared to samples without any marker.

Further, Table 12 shows that 16/34 markers from Table 1 were detected in 10 endometrium cancer samples, which displayed 26 markers altogether. Several markers of Table 1 were present in 2 samples (UPRT, ARHGAP35) or 3 samples (ASCC3, GRM5, HTR2A, MS4A8) and may therefore be promising markers for the detection of elevated TMB in endometrium cancer. As reported in Table 11, several markers can frequently occur together, and also in the current experiment ASCC3 and FUBP1 occurred together in sample no. 3.

TABLE 11

| Sample | No. | | | | Variant No. | Allelic |
|---|---|---|---|---|---|---|
| No. | variants | Gene | position in GRCh37/hg19 | | in Table 1 | Frequency (%) |
| 1 | 275 | PCDH19 | chrX | 99662008 | 3 | 13.75 |
| 2 | 281 | RALA | chr7 | 39745749 | 30 | 9.26 |
| 3 | 350 | ASCC3 | chr6 | 101296418 | 15 | 3.37 |
| | | FUBP1 | chr1 | 78428511 | 25 | 3.62 |
| 4 | 48 | None | | | | |
| 5 | 324 | UPRT | chrX | 74519615 | 6 | 13.03 |
| 6 | 419 | BRWD3 | chrX | 79942391 | 27 | 4.35 |
| | | GRM5 | chr11 | 88338063 | 23 | 11.2 |
| | | HTR2A | chr13 | 47409732 | 10 | 9.51 |
| | | MS4A8 | chr11 | 60468341 | 11 | 7.92 |
| 7 | 116 | HTR2A | chr13 | 47409732 | 10 | 3.8 |
| 8 | 18 | None | | | | |
| 9 | 24 | None | | | | |
| 10 | 24 | None | | | | |
| 11 | 70 | None | | | | |
| 12 | 27 | None | | | | |
| 13 | 23 | None | | | | |
| 14 | 33 | None | | | | |
| 15 | 73 | ASCC3 | chr6 | 101296418 | 15 | 3.5 |
| 16 | 113 | None | | | | |
| 17 | 285 | ARHGAP35 | chr19 | 47424921 | 1 | 5.7 |
| | | CUL4B | chrX | 119678368 | 28 | 6.34 |
| 18 | 381 | ASCC3 | chr6 | 101296418 | 15 | 9.58 |
| | | GRM5 | chr11 | 88338063 | 23 | 3.39 |
| | | HTR2A | chr13 | 47409732 | 10 | 5.88 |
| | | MS4A8 | chr11 | 60468341 | 11 | 7.27 |
| | | ZNF236 | chr18 | 74635035 | 19 | 6.59 |
| 19 | 17 | None | | | | |
| 20 | 43 | None | | | | |
| 21 | 26 | None | | | | |
| 22 | 14 | None | | | | |
| 23 | 212 | None | | | | |
| 24 | 15 | None | | | | |
| 25 | 16 | None | | | | |
| 26 | 130 | None | | | | |
| 27 | 49 | None | | | | |
| 28 | 16 | None | | | | |
| 29 | 12 | None | | | | |
| 30 | 12 | None | | | | |
| 31 | 65 | None | | | | |
| 32 | 34 | None | | | | |
| 33 | 24 | None | | | | |
| 34 | 64 | ALG13 | chrX | 110970087 | 12 | 30.91 |
| | | ARHGAP35 | chr19 | 47424921 | 1 | 9.1 |
| | | COL14A1 | chr8 | 121228689 | 7 | 40.36 |
| | | GRM5 | chr11 | 88338063 | 23 | 5.35 |
| | | MBOAT2 | chr2 | 9098719 | 17 | 45.26 |
| | | MS4A8 | chr11 | 60468341 | 11 | 56.14 |
| | | UPRT | chrX | 74519615 | 6 | 14.23 |
| 35 | 56 | None | | | | |
| 36 | 29 | None | | | | |

We claim:

1.-27. (canceled)

28. A composition comprising:
primer pairs configured for the amplification of a plurality of different target sequences in a subject nucleic acid sample, wherein the target sequences comprise at least a subset of the loci listed in Table 1.

29. The composition of claim 28, further comprising:
reagents for sequencing amplicons generated by the primer pairs.

30. The composition of claim 28, comprising a cartridge, wherein the primer pairs are within the cartridge.

31. The composition of claim 29, comprising a cartridge, wherein the primer pairs and reagents for sequencing amplicons are within the cartridge.

32. The composition of claim 28, further comprising:
primer pairs configured for amplification of at least a portion of the catalytic subunit of polymerase ε (POLE) gene sequence.

33. A composition comprising:
a panel, the panel comprising a plurality of nucleic acid probes, the probes optionally linked to a solid support, wherein the nucleic acids probes hybridize to a plurality of target sequence, the target sequences comprising at least a subset of loci listed in Table 1.

34. The composition of claim 34, wherein the composition comprises a cartridge, wherein the probes are within the cartridge.

34. The composition of claim 33, further comprising at least one POLE nucleic acid probe, optionally linked to a solid support, wherein the at least one POLE nucleic acid probe hybridize to at least a portion of the POLE gene sequence.

35. A method comprising:
(a) contacting a patient sample nucleic acid sample with the composition of claim 1;
(b) amplifying the nucleic acid to generate amplicons;
(c) sequencing the amplicons to generate sequence data; and
(d) analyzing the sequence data to identify amplicons comprising a mutation listed in Table 1.

36. The method of claim 35, wherein the method is performed in a cartridge.
